(19) United States
(12) Patent Application Publication
(10) Pub. No.: US 2015/0072867 A1
(43) Pub. Date: Mar. 12, 2015

(54) ANALYSIS OF NUCLEIC ACID MOLECULES DISTRIBUTED ON A SURFACE OR WITHIN A LAYER BY SEQUENCING WITH POSITION IDENTIFICATION

(71) Applicant: MAX-PLANCK-GESELLSCHAFT ZUR FÖRDERUNG DER WISSENSCHAFTEN E.V., Munich (DE)

(72) Inventors: Aleksey Soldatov, Berlin (DE); Tatiana Borodina, Berlin (DE); Hans Lehrach, Berlin (DE)

(73) Assignee: MAX-PLANCK-GESELLSCHAFT ZUR FÖRDERUNG DER WISSENSCHAFTEN E.V., Munich (DE)

(21) Appl. No.: 14/389,907

(22) PCT Filed: Apr. 3, 2013
(86) PCT No.: PCT/EP2013/057053

§ 371 Date: Oct. 1, 2014

(30) Foreign Application Priority Data
Apr. 3, 2012 (EP) 12163055.2

(51) Int. Cl. C12Q 1/68

(52) U.S. Cl. CPC C12Q 1/6874
USPC 506/2; 435/6.11

(57) ABSTRACT

The present invention describes a method for identification of areas of a sample from which nucleic acid molecules originate using labeling of said nucleic acid molecules by two-dimensionally distributed oligonucleotide markers. Further analysis of hybrids between the nucleic acid molecules and the oligonucleotide markers allow identification of the original position of the labelled nucleic acid molecules in the sample.

Many molecular biology methods can't be used for the analysis of nucleic acid (NA) molecules in situ. They require NA molecules to be in solution. The present invention describes a method for preserving information about original spatial distribution of nucleic acid molecules transferred from a surface or a layer into solution. We suggest labelling of nucleic acid molecules using two-dimensionally distributed oligonucleotide markers. Further analysis of oligonucleotide markers allow to identify the original position of labelled nucleic acid molecules. The suggested method is useful for expression profiling and locus specific sequencing in tissue sections.

### BACKGROUND OF THE INVENTION

Biological processes are spatially organized. They rely upon the interplay of many different components forming an intricate structure of cells, tissues and organisms. Molecules participating in these processes have a certain spatial distribution. Understanding the biological processes is critically dependent on a detailed knowledge of this distribution.

Objects with two-dimensional distribution of nucleic acid molecules, for example tissue sections, are widely studied. There exist methods for nucleic acid analysis in tissue sections, for example in situ hybridization or in situ PCR. However, not all molecular biology methods are applicable when working with tissue sections.

Two-dimensional tissue sections are convenient objects to study distribution of molecules. Several sequential sections restore a 3D spatial location of molecules. However, many molecular biology methods, for example sequencing, cannot be performed directly in tissue sections. It would be advantageous to be able to transfer molecules from the tissue section to another surface or into solution, where appropriate methods of analysis could be performed. However, such transfer raises questions of keeping information about initial distribution of the nucleic acid molecules.

Replication of 2D distributed objects (nucleic acid molecules, cells) has been long used in molecular biology. Main purposes are to perform analysis which is not possible with original sample and (ii) multiplying 2D sample for several analyses.

Southern and Northern methods are known, wherein nucleic acid molecules are transferred from gel to membrane. Membrane allows analyzing transferred molecules by hybridization preserving the relative distribution they had in gel. Replica of DNA of library clones on membranes is used to search for particular clones using hybridization. Replication of bacterial colonies to other plates allows analyzing in parallel, for example, their resistance to several antibiotics.

In the last decade, two methods were suggested for multiplying nucleic acid arrays by replication. In one approach nucleic acid array features are first amplified on the array, then the array with amplified features is brought into tight contact with transfer support, to which parts of amplified molecules are transferred and get covalently attached (U.S. Pat. No. 7,785,790). In the nanostamping approach nucleic acid molecules hybridised to sample surface are brought into direct contact with capturing groups on the target surface. Chemical binding with the target surface is stronger than hybridization and after separating surfaces, nucleic acid molecules remain on the target surface (U.S. Pat. No. 7,862,849).

The general principle of replication is bringing into contact a surface with 2D distributed nucleic acid molecules with a target surface, to which they are transferred by diffusion or direct contact. So far nucleic acid molecules have been transferred to surfaces were they were captured either physically (stuck in gel) or by chemical bonds (covalent, ion exchange, affinity) involving certain reactive groups on the nucleic acid molecules and on the target surface, but not involving the nucleotide sequence of the molecules.

Objective of the present invention is to provide a method capable of preserving the information about spatial distribution of nucleic acid molecules transferred from a surface to another surface.

This objective is solved by the present methods as shown below. Further preferred embodiments of the present invention are disclosed in the dependent claims, the description, the figures and the examples.

Surprisingly it was found that methods according to the present invention allow the transfer of nucleic acid molecules to another surface preserving information about their original positions.

### DESCRIPTION OF THE INVENTION

Many molecular biology methods, for example sequencing, cannot be performed directly in tissue sections. Besides, even if in situ sequencing would be feasible there is another problem: uneven distribution of analyzed molecules within a sample. Some regions of tissue section does not contain molecules for the sequencing, whereas others regions—too much. Empty regions make in situ sequencing expensive and ineffective, while overcrowded regions may be completely not suitable for in situ sequencing, because it would not be possible to distinguish individual molecules. Molecular density problem can't be solved without redistribution of molecules. To make redistribution feasible some method of preserving information about the original positions of molecules should be elaborated.

In the present invention it is suggested to attach oligonucleotide labels or markers to nucleic acid molecules before changing their relative positions. The labels bear information about positions of the nucleic acid molecules (FIG. 1). This approach permits not only redistribution, but any other manipulations, which keep the label, which consists of or contains a known sequence, on the nucleic acid molecule. It is possible to mix molecules together, put them into solution, perform enzymatic reactions: as long as the label remain associated with nucleic acid molecule it is possible to identify its original position. The standard optimized methods for preparation of sequencing libraries and for sequencing may be applied for analysis of the labeled nucleic acid molecules.

In the present invention it is suggested to use two-dimensionally distributed oligonucleotide markers for labeling of the nucleic acid molecules. Current technologies (microarrays, distributed microbeads) permit to distribute oligonucleotides with high density on a small surface and provide high spatial resolution of oligonucleotide markers for labeling. Besides, two-dimensionally distributed oligonucleotide markers may be transferred to the nucleic acid molecules in a sample all at once in parallel.

In the present invention it is suggested to use hybridization for association of oligonucleotide markers and distributed nucleic acid molecules. Hybridization is strong, very specific, does not require modification of nucleic acid molecules, and is convenient for subsequently covalent linking of oligonucleotides and distributed nucleic acid molecules by ligation or primer extension.

The present invention is directed to methods for preserving information about original spatial distribution of nucleic acid molecules transferred from a surface to another surface or into solution.

This is accomplished by the use of hybridization of nucleic acids for creating replicas of nucleic acids molecules or molecular complexes containing nucleic acid molecules located either on a surface of a sample or within a sample, wherein said creating replicas is obtaining on a target surface the relative distribution of nucleic acid molecules resembling the original distribution. During the inventive method the nucleic acid molecules of a sample remain on their original positions relatively to each other but move perpendicular to an overlying target surface.

Hybridization as a way to capture nucleic acid molecules makes the replication or preparation of a replica highly selective, since only nucleic acid molecules having complementary sequences will be hold on the target surface. Besides, hybridization is a controllable process and allows regulation of the time of replication and, consequently, the number of transferred nucleic acid molecules. The method does not require direct contact of 2D distributed nucleic acid molecules to th binding sites on the target surface. This means that (i) the transfer may be performed between large solid surfaces, which can't form uniform tight contact and (ii) the method may be applied to transfer nucleic acid molecules from 3D samples to the target surface.

The term “replica” as used herein refers to a copy of the distribution of nucleic acids with preservation of their original distribution to a target surface by hybridization. The target surface with the transferred nucleic acids held by hybridization with preservation of their original distribution is the created replica. Thus, replica is obtaining on a target surface the relative distribution of nucleic acid molecules or molecular complexes containing nucleic acid molecules resembling the original distribution.

One preferred replication method according to the invention comprises the following steps:
a) providing sample with nucleic acid molecules located either on a surface or within a layer;
b) providing target surface with nucleic acid molecules, capable to hybridization-based binding to nucleic acid molecules from (a);
c) (option 1) if nucleic acid molecules are not attached to the sample, providing conditions to minimize shift of molecules from the original positions;
d) (option 2) if nucleic acid molecules are attached to the sample, providing conditions for gradual releasing of nucleic acid molecules;
e) assembling sample with nucleic acid molecules from (a) against the target surface from (b) with solution in between, such that nucleic acid molecules from the sample can reach target surface by diffusion through solution;
f) (option 3) if nucleic acid molecules are attached to the sample, providing conditions for releasing nucleic acid molecules from the original positions in the sample,
g) providing conditions for diffusion of nucleic acid molecules from the sample to the target surface and hybridization-based binding of nucleic acid molecules from the sample to the nucleic acid molecules on the target surface;
h) (optional) providing conditions for slowing down the formation of new hybrids of nucleic acid molecules;
i) disassembling the sample (a) and target surface (b).

The present invention refers further to a method for identification of areas of a sample from which nucleic acid molecules originated using labeling of said nucleic acid molecules by two-dimensionally distributed oligonucleotide markers comprising the following steps:
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if nucleic acid molecules are not attached to the sample, providing conditions to minimize shift of nucleic acid molecules from the original positions on or within the sample; or
c′) if nucleic acid molecules are attached to the sample, providing conditions for releasing the nucleic acid molecules;
d) assembling the sample and the target surface in such a way, that the distance from positions of said nucleic acids to the target surface is smaller than the distortion acceptable for the replica and with a medium in between sample and target surface;
e) providing conditions for diffusion of the nucleic acid molecules from the sample to the target surface and hybridization-based binding of the nucleic acid molecules to the oligonucleotide markers on the target surface;
f) releasing of hybrids of the nucleic acid molecules and the oligonucleotide markers into solution;
   or
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if oligonucleotide markers are not attached to the target surface, providing conditions to minimize shift of nucleic acid oligonucleotide markers from the original positions on the target surface; or
c′) if oligonucleotide markers are attached to the target surface, providing conditions for releasing of oligonucleotide markers;
d) assembling the sample of and the target surface in such a way, that the distance from positions of oligonucleotide markers to the sample is smaller than the distortion acceptable for the replica and with a medium in between sample and target surface;
e) providing conditions for diffusion of the oligonucleotide markers from the target surface to the sample and hybridization-based binding of the oligonucleotide markers to the nucleic acid molecules on the sample;
f) releasing of hybrids of nucleic acid molecules and the oligonucleotide markers into solution;
   or
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if the oligonucleotide markers and/or the nucleic acid molecules are not attached to the sample, providing conditions to minimize shift of the oligonucleotide markers and/or the nucleic acid molecules from the original positions; or
c′) if the oligonucleotide markers and/or the nucleic acid molecules are attached to the sample, providing conditions for releasing of the oligonucleotide markers and/or the nucleic acid molecules;
d) assembling the sample and the target surface in such a way, that the distance from positions of said nucleic acids to the target surface is smaller than the distortion acceptable for the replica and with a medium in between sample and target surface;
e) providing conditions for diffusion of the oligonucleotide markers from the target surface and the nucleic acid molecules from the sample and hybridization-based binding of the marker oligonucleotides to the nucleic acid molecules within the medium between sample and target surface;
f) disassembling the sample and the target surface and collecting the medium containing hybrids of the nucleic acid molecules and the oligonucleotide markers; and
g) analyzing the hybrids in order to determine the nucleotide sequence of the oligonucleotide markers;
h) identification of the areas of the sample from which the nucleic acid molecules originated.

In another embodiment of the inventive method step c′) is performed after step d) (assembling of sample and target surface) without disturbing the assembly (which means before disassembling). Hence, releasing of the nucleic acid molecules and/or the oligonucleotide marker may be carried out before or after assembling the sample and the target surface (step d)). Releasing of the nucleic acid molecules may be done by several ways:

One possibility would be increasing of the temperature if the attachment or binding of the nucleic acid molecules to the sample and/or the oligonucleotide markers to the target surface is temperature-sensitive. Another preferred way is introducing of a cleavage agent by changing the medium between the sample and the target surface in the assembly. This is for example possible if the sample or the target surface or both are permeable for liquids. Another preferred way is using lighting wherein the nucleic acid molecules are held on the original positions in the sample by photocleavable binding and wherein either the sample or the target surface are transparent for the light having required wavelength.

One object of the current invention wherein hybridization of the nucleic acid molecules with the oligonucleotide markers is carried out on the target surface is a method for identification of areas of a sample from which nucleic acid molecules originate using labeling of said nucleic acid molecules by two-dimensionally distributed oligonucleotide markers comprising the following steps:
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if nucleic acid molecules are not attached to the sample, providing conditions to minimize shift of nucleic acid molecules from the original positions on or within the sample; or
c′) if nucleic acid molecules are attached to the sample, providing conditions for releasing the nucleic acid molecules;
d) assembling the sample and the target surface in such a way, that the distance from positions of said nucleic acids to the target surface is smaller than the distortion acceptable for the replica and with a medium in between sample and target surface;
e) providing conditions for diffusion of the nucleic acid molecules from the sample to the target surface and hybridization-based binding of the nucleic acid molecules to the oligonucleotide markers on the target surface;
f) releasing of hybrids of the nucleic acid molecules and the oligonucleotide markers into solution;
g) analyzing the hybrids in order to determine the nucleotide sequence of the oligonucleotide markers;
h) identification of the areas of the sample from which the nucleic acid molecules originated.

Another object of the current invention wherein hybridization of the nucleic acid molecules with the oligonucleotide markers is carried out on the surface of the sample or in the sample is a method for identification of areas of a sample from which nucleic acid molecules originate using labeling of said nucleic acid molecules by two-dimensionally distributed oligonucleotide markers comprising the following steps:
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if oligonucleotide markers are not attached to the target surface, providing conditions to minimize shift of nucleic acid oligonucleotide markers from the original positions on the target surface; or
c′) if oligonucleotide markers are attached to the target surface, providing conditions for releasing of oligonucleotide markers;
d) assembling the sample of and the target surface in such a way, that the distance from positions of oligonucleotide markers to the sample is smaller than the distortion acceptable for the replica and with a medium in between sample and target surface;
e) providing conditions for diffusion of the oligonucleotide markers from the target surface to the sample and hybridization-based binding of the oligonucleotide markers to the nucleic acid molecules on the sample;
f) releasing of hybrids of nucleic acid molecules and the oligonucleotide markers into solution;
f) disassembling the sample and the target surface and collecting the medium containing hybrids of the nucleic acid molecules and the oligonucleotide markers; and
g) analyzing the hybrids in order to determine the nucleotide sequence of the oligonucleotide markers;
h) identification of the areas of the sample from which the nucleic acid molecules originated.

The present invention refers further to a method for identification of areas of a sample from which nucleic acid molecules originate using labeling of said nucleic acid molecules by two-dimensionally distributed oligonucleotide markers in the medium between the target surface and the sample comprising the following steps:
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if the oligonucleotide markers and/or the nucleic acid molecules are not attached to the sample, providing conditions to minimize shift of the oligonucleotide markers and/or the nucleic acid molecules from the original positions; or
c′) if the oligonucleotide markers and/or the nucleic acid molecules are attached to the sample, providing conditions for releasing of the oligonucleotide markers and/or the nucleic acid molecules;
d) assembling the sample and the target surface in such a way, that the distance from positions of said nucleic acids to the target surface is smaller than the distortion acceptable for the replica and with a medium in between sample and target surface;
e) providing conditions for diffusion of the oligonucleotide markers from the target surface and the nucleic acid molecules from the sample and hybridization-based binding of the marker oligonucleotides to the nucleic acid molecules within the medium between sample and target surface;
f) disassembling the sample and the target surface and collecting the medium containing hybrids of the nucleic acid molecules and the oligonucleotide markers; and
g) analyzing the hybrids in order to determine the nucleotide sequence of the oligonucleotide markers;
h) identification of the areas of the sample from which the nucleic acid molecules originated.

The present invention refers also to the above described methods comprising step c) and step c′). This can be necessary if some nucleic acid molecules and/or oligonucleotide markers are attached and other ones are not attached. It is especially possible that the nucleic acid molecules are not attached but the oligonucleotide markers are attached. In this case it is preferred that step c′) is performed after step d), hence after the assembling but before disassembling. With this succession of steps it is ensured that the shift of the nucleic acid molecules and/or oligonucleotide markers which are not attached is minimized before the assembly and additionally the nucleic acid molecules and/or oligonucleotide markers which are attached can be migrate.

The present invention refers to a method for identification of areas of a sample from which nucleic acid molecules originate using labeling of said nucleic acid molecules by two-dimensionally distributed oligonucleotide markers. The same steps a) to h) may also be comprised by a method for analyzing the distribution of nucleic acid molecules within a sample or on the surface of a sample by hybridization of nucleic acid molecules with oligonucleotide markers. Furthermore the present invention refers to a method for analyzing the relative distribution of nucleic acid molecules within a non-fluidic sample by labeling the nucleic acid molecules with oligonucleotide markers attached to a target surface and wherein their spatial distribution on the target surface is known. Another preamble for the methods according to the invention could read as follows: Method for identification of nucleic acid molecules located in defined areas of a sample by hybridization of the nucleic acid molecules with oligonucleotide markers attached to and thereby defining corresponding areas on a target surface.

The nucleic acid molecules can be either located on the surface of the sample or within a sample. Preferably, the nucleic acid molecules located on a surface of the sample provided in step a) are distributed in a nucleic acid array or protein array, and the nucleic acid molecules distributed within a sample are distributed in a gel layer, in tissue section, in cell or tissue array or in block of tissue. For example, the nucleic acids can be contained in a gel and can be mobilized out of the gel to the surface of the gel. Alternatively, the nucleic acids can be provided on the surface of a glass slide.

The sample with nucleic acid molecules also comprises nucleic acid molecules that are hybridized to the nucleic acids in the sample. This means that nucleic acid molecules could be distributed on the surface of the sample or within the sample and to this nucleic acids further nucleic acids are hybridized. Thus providing a sample with nucleic acid molecules located either on a surface or within a sample also includes hybridization products of nucleic acid molecules. Consequently, the term nucleic acid molecules also comprise hybridization products of nucleic acids.

The target surface comprises a plurality of at least one type of oligonucleotides attached to the target surface. The target surface can be of any texture. The target surface should be covered with oligonucleotide markers, at least in the area to which the transfer is performed. Transferred nucleic acid molecules hybridize preferably directly to the oligonucleotide markers immobilized on the target surface.

A lot of ways are known in the prior art for preparation of target surfaces covered with oligonucleotides, e.g. spotting, on-surface synthesis, attaching to beads, fixation in gel, etc.

The nucleic acid molecules may be either attached or not attached to the sample. If the nucleic acid molecules are not attached to the sample it is preferred to apply conditions to minimize the shift of nucleic acid molecules from their respective original positions. Such conditions could be a decrease of temperature. Preferably, the temperature is decreased below 24° C., preferably below 20° C., more preferably below 16° C., preferably below 12° C., even more preferred below 8° C., and more preferred below 4° C.

On the other hand if the nucleic acid molecules are attached to the sample it may be advisable to apply conditions, wherein I release of the nucleic acid molecules in the sample occurs. The nucleic acid molecules may be attached to the sample, for example by hybridization to complementary sequences covalently bound to the sample, or through cleavable groups.

In one embodiment the nucleic acid molecules in the sample are held on the original positions by chemical- or enzyme-sensitive binding. Thus, before or after assembling the sample and the target surface, conditions can be applied, wherein release of the nucleic acid molecules from the sample and/or oligonucleotide markers from the target surface occurs. Said conditions for release of nucleic acid molecules and/or oligonucleotide markers may be addition of a cleavage agent which acts slow enough to ignore those molecules which change the position before assembling the sample and the target surface

In one embodiment said low activity of the cleavage agent is provided by decreasing concentration of said agent or by providing reaction conditions decreasing the activity of said agent.

Diffusion of nucleic acid molecules within the sample may be physically hindered by a surrounding matrix, for example an agarose or acrylamide gel. In this case diffusion exists but it is very slow: the time of appearing of free molecules on the surface of the sample is much longer than the time of assembling the sample and the target surface. It is possible to assemble the sample and the target surface and wait till the nucleic acid molecules diffuse enough to reach the target surface. It might be possible to speed up the diffusion by raising the temperature during step e). Nucleic acid molecules may be just physically stuck within the sample, for example in gel after gel electrophoresis.

The sample with nucleic acid molecules and the target surface may be assembled under conditions where the nucleic acid molecules do not leave their relative positions on the sample surface. Such conditions can comprise e.g. low temperature, a filter or net between the surfaces, enzymes and/or chemical substances preventing detachment at this stage or vice versa the lack of such enzymes and/or chemical substances needed for detachment.

Preferably the sample with the nucleic acid molecules and the target surface are assembled under “wet” conditions meaning that the sample and target surface are surrounded by solution, i.e. liquid and/or that liquid is between both surfaces. Both surfaces are arranged such that both surfaces come into contact with each other in a sandwich-like configuration. A thin liquid film can preferably exist between both surfaces. The liquid between the surfaces and/or around the assembled sandwich-like configuration can comprise enzymes and/or chemical substances needed e.g. for detachment. If a filter or net between the surfaces is used during assembly, such a net would prevent direct contact of the surfaces.

The surfaces in the sandwich-like configuration shall be tightly pressed to each other to make the distance between the surfaces so that the distance between both surfaces is so small that no blurring of the distribution pattern occurs. Assembling such sandwich-like configurations is performed as shown in FIG. 3 and is well known to the skilled artisan and corresponds mutatis mutandis to the procedures known from e.g. western/northern blotting. Surface assembly would be done preferably at room or lower temperature, so that the nucleic acid molecules do not go off the sample surface. Generally, the inventive method does not require a direct contact between the nucleic acids distributed on the sample and the oligonucleotides on the target surface. This means that transfer may be performed between large solid surfaces, which can't form uniform tight contact.

The terms “sandwich-like configuration” or “assembly” both refer to the configuration that the sample and the target surface are stacked with a medium in between. This means the sample and the target surface are one above the other but between both surfaces a medium is located. The term “sample” as used herein refers to an object with a two or three-dimensional distribution of nucleic acid molecules. Thereby the consistence of the sample has to be in such a way that the nucleic acid molecules of interest have an inhomogeneous or unequal distribution which is preferably not highly variable. Thus, the nucleic acids should not be in solution. Preferred samples are non-fluidic, gel-like, fixated or solid. Examples of suitable samples are tissue sections, tissue blocks, a gel layer, a cell, a cell layer, a tissue array, yeasts or bacteria on a culture plate, membrane, paper or fabric, or a carrier with spots of isolated or synthetic nucleic acid molecules. In general the sample may comprise a carrier made of glass, plastic, paper, a membrane (eg nitrocellulose) or fabric. For example a tissue section is usually applied on a glass slide. A cell layer could also be provided on a glass slide or on a plastic dish. Unicellular organisms may be provided on culture plates, on filter paper or on a fabric. The nucleic acid molecule may be within the sample for example within a fixed cell, within a gel or within a tissue. Alternatively the nucleic acid molecules may be provided on the surface of a sample like a microarray (2D array on a solid substrate; usually a glass slide or silicon thin-film cell), preferably a DNA array also commonly known as DNA chip or biochip. Most preferable the sample is a tissue section. Said tissue section but also other samples (eg cells or unicellular organisms) may be frozen, (fresh frozen or fixed frozen) fixed (formaldehyde fixed, formalin fixed, acetone fixed or glutaraldehyde fixed) and/or embedded (using paraffin, Epon or other plastic resin). Such tissue sections like can be prepared with a standard steel microtome blade or glass and diamond knives as routinely used for electron microscopic sections. Furthermore small blocks of tissue (less than 15 mm thick) can be processed as whole mounts. In case the nucleic acid molecules are on the surface of the sample, thickness of the sample does not really matter so that any thickness could be used. In case the nucleic acid molecules are located within the sample like tissue slides, thickness should be in a range that the nucleic acid molecules could move out of the sample to the target surface. A preferred thickness of such samples is for example 1 μm to 1 mm and preferably 5 μm to 10 μm.

The term “medium” as used herein refers to any material which allows nucleic acid molecules to diffuse through. Hence the term “medium” includes solutions, gels as well as other viscous or honey-like materials. Most preferably the medium used within the inventive method is a solution which may be an aqueous solution like a buffer, preferably on basis of PBS-buffers (Phosphate buffered saline) as well as Tris- and triethanolamine buffers (TE-buffer). It is further preferred that the pH-value of the used medium prevents denaturation of the nucleic acid molecules. Hence the pH of the medium or buffer is most preferably adjusted around 7.5 for RNA and around 8.0 for DNA. The medium or solution may further comprise some additives like cleavage agents (enzymes) or inhibitors of RNase or Dnase. Thereby the medium in the assembly of the sample and the target surface can also be emitted by the sample or the target surface. For example if the sample is a gel or contains a gel on the surface the medium may be a thin liquid film which is generated when some liquid leaks out of the gel due to some pressure during the assembling of the sample and the target surface.

The medium used in the inventive method should be chosen such that the nucleic acid molecules from the sample can reach the target surface by diffusion through the medium. The medium is used for diffusion of nucleic acid molecules from the sample to the target surface. This medium is preferably a liquid layer. Viscosity of the liquid layer may be increased to minimize the liquid flow along the target surface, for example, by inclusion of polymer molecules into the liquid. In the extreme case, those polymers may form a gel, which completely prevents the liquid flow, but preserves a possibility to nucleic acid molecules to diffuse from the sample to the target surface.

Step d), assembling the sample and the target surface with a medium in between comprises that the target surface is placed on top of (or below, depending on the direction of the transfer) the sample wherein the medium is added to the sample or to the target surface before. Assembling of the sample and the target surface in step d) is preferably done in such a way, that the distance from positions of the nucleic acid molecules on the surface of the sample or within the sample to the target surface is smaller than the distortion acceptable for the replica. This means if the tolerable or acceptable distortion is less than 1 mm the distance between the sample and the target surface should most preferably be less than 1 mm. However this is a question of resolution and in case a high resolution is desired, the distance between sample and target surface should be less or much less than the distortion. Since the degree of distortion is a question of resolution provided by the inventive methods, step d) in all methods disclosed herein could also read as follows:
d) assembling the sample and the target surface with a medium in between sample and target surface in a way that the distance between sample and target surface is minimized;
   or step d) in all methods disclosed herein could alternatively read as follows:
d) assembling the sample and the target surface with a medium in between sample and target surface in a way that the distance of each nucleic acid molecule to the target surface or of each oligonucleotide marker to the sample is less than the distortion of the respective nucleic acid molecule;
   or step d) in all methods disclosed herein could alternatively read as follows:
d) assembling the sample and the target surface with a medium in between sample and target surface in a way that the distance each nucleic acid molecule or each oligonucleotide marker has to move in straight direction to the target surface is less than the distance the respective nucleic acid molecule or oligonucleotide marker is allowed to move straight in a direction perpendicular to the direction which is straight to the target surface.

However since the distortion is only an aspect how accurate the obtained data are but not whether the methods disclosed herein work, step d) could in all methods disclosed herein also simplified as follows:

d) assembling the sample and the target surface with a medium in between sample and target surface

or like

d) assembling the sample and the target surface with a medium in between sample and target surface so that the nucleic acid molecules can move to the target surface or so that the oligonucleotide markers can move to the sample or so that the nucleic acid molecules and the oligonucleotide markers can move into the medium.

The term “distortion” can also be explained as the drift of the nucleic acid molecules.

If the sample consists or comprises of a layer the maximal possible distance of the nucleic acid molecules in the sample to the target surface should be smaller than the distortion acceptable for the replica. Therefore the distance from the surface of the layer not facing the target surface (or the bottom side) is relevant. “Distortion” as used herein denotes the alteration of the original, relative distribution of the nucleic acid molecules during the inventive method. One aim of the inventive method to avoid distortion or at least to lessen it till a tolerable extent.

Furthermore the medium used prevent the direct contact of the sample and the target surface, which is important for prevention of contamination of the target surface because of unspecific binding. Of course a direct contact of the sample and the target surface should also be avoided during assembling and disassembling of the sample and the target surface.

“Two-dimensionally distributed oligonucleotide markers” as used herein refers to immobilization of a variety of oligonucleotide marker on a target surface or in a target (if the target is for example a gel) forming a stable pattern wherein the oligonucleotide marker are covalently or not covalently linked to the target surface. The immobilization therefore refers preferably to association of oligonucleotide markers to the target by covalent bonding or non covalent interaction between the oligonucleotide marker and the target. Possible non-covalent interactions are: hydrogen bonds, ionic bonds, van der Waals forces, and hydrophobic interactions. Many polymers, such as polystyrene and polypropylene are hydrophobic in nature. Nevertheless there are also manufacturers which supply targets having specialized surfaces optimized for different adhesion conditions. The covalent or non covalent bonding may also be indirect. “Indirect covalent bonding” as used herein refers to immobilization of oligonucleotide marker wherein the oligonucleotide markers are covalently linked to a second compound which mediates the immobilization to the target. A suitable target may be made from glass, plastic, paper, membrane or a gel, like agarose gel.

However, immobilization, especially using indirect covalent bonding, may also occur by strong adhesion. Thus, an effective immobilization according to the present invention may be realized not only by chemical bonding, but also by immobilization related to physisorption.

Furthermore the term “two-dimensionally distributed oligonucleotide markers” refers to oligonucleotide markers immobilized on a target surface having a defined distribution within the plane on the target surface. Thereby each type of oligonucleotide marker (having a specific known sequence) represents or identifies one specific area or region on the target surface. The form (quadrates, concentric circles) as well as the size of the areas or regions is freely selectable and should be adapted to the sample and to the specific problem which should be solved with the individual scientific example using the inventive method.

In one exemplary version the target surface is divided into 100 areas or regions with a square configuration, ten per row and ten per line (comparable to a chessboard). In this example 100 known sequences as part of the oligonucleotide markers are needed, wherein the oligonucleotide markers of each area contain all the same known sequence and represent or identify this specific area. Alternatively each area with a square configuration is identified by the combination of two oligonucleotide markers, wherein one oligonucleotide marker can bind to the 3′ end and the other can bind to the 5′ end of a nucleic acid molecule. On can also say that each area with a square configuration is identified by the overlapping of bigger areas (here rows and lines). In this case the number of oligonucleotide markers and known sequences needed is smaller because one sequence may identify the line and another sequence may identify the row (20 compared to 100 different sequences). Before the assembling is carried out the target surface and the sample have to be marked so that later during analysis it can be reproduce which area on the target surface (and respectively known sequence) corresponds to which area on or in the sample. One possibility is to mark one corner on the target sample and one the target surface which will be congruent in the assembly (see FIG. 2 or 11).

In general, there is a continuous spectrum how strong nucleic acid molecules may be held in a sample—from a strong covalent attachment of nucleic acids to the sample to a very loose association.

The term “not-attached to the sample”, as used herein, means that after the preparation of the sandwich-like assembly of the sample and the target surface, the nucleic acid molecules will leave the sample and reach the target surface without any supportive action only by diffusion. This means the nucleic acid molecules are not covalently bound to the sample but are associated in a way that they cannot freely change their position within the sample. In the contrary the inventive method comprises conditions to minimize a shift or more general the free movement (especially the lateral movement) of these nucleic acid molecules to minimize the distortion. The term “attached to the sample”, as used herein, means that after the preparation of the sandwich-like assembly of the sample and target surface, nucleic acid molecules would not leave the sample without assistance, which could be because of fixation of the sample or because of a covalent linkage of the nucleic acid in the sample or on the sample surface. Within the inventive method the nucleic acid molecules which are attached have to be released from the sample before or preferably after assembling (during step c′)). This may be done by different cleavage agents (like enzymes), light, but also by a change in pH or temperature.

The incubation time of the assembly is dependent from many variables, such as accessibility of the nucleic acids in the sample, incubation temperature and other factors. Generally, the incubation time should be long enough to allow sufficient hybridization, but still short enough to prevent e.g. unspecific binding. Under aspects of process economy, the incubation time should be chosen to be as short as possible. The skilled artisan can determine the optimal incubation time with minimum routine experimentation.

Step e) of the inventive method refers to incubating the assembly of the sample and the target surface of step d) under conditions sufficient to allow diffusion or migration of the nucleic acid molecules from the sample to the target surface and subsequently allow hybridization of the nucleic acids to the immobilized oligonucleotides. These conditions are explained in more detail above. During the inventive method lateral movements of the nucleic acids are suppressed so that the term “diffusion” or “migration” of the nucleic acid molecules in step e) refers only to a movement of the nucleic acid molecules primarily along a perpendicular axes. Thus the nucleic acid molecules leave the sample on a vertically way, on the direct route, to the target surface so that on the surface of the target a copy or replica is created which contains the nucleic acid molecules in an unaltered relative distribution or at least in a relative distribution with a minimal distortion.

Detachment conditions (certain temperature, light, solution) may be applied to the assembly of the sample with distributed nucleic acid molecules and the target surface. Temperature may be applied to release the nucleic acid molecules or the oligonucleotide markers if the binding to the sample is temperature-sensitive. Thus, in one embodiment the condition for releasing the nucleic acid molecules from the original positions in the sample occurs by increasing the temperature.

In another embodiment the nucleic acid molecules are held on the original positions in the sample by temperature-sensitive binding by hybridization or through thermolabile covalent bonds, abasic site or formaldehyde linkage. Detachment can also occur by providing a thermoactivated cleavage agent, enzyme or chemical reagent in the solution between the sample and the target surface.

Hence, in another embodiment the condition for releasing the nucleic acid molecules from the original positions in the sample or releasing the oligonucleotide markers from the target surface is changing the solution between the sample and the target surface.

The possibility to change solution in the contact area in the assembly substantially increases the variants of nucleic acid molecules attachment to the sample, and consequently, types of samples. If nucleic acid molecules are attached to the nucleic acid sample by hybridization to a complementary sequence, duplex may be denatured by changing the pH or ionic strength of the solution, or changing the solution to the one decreasing the denaturation temperature (like formamide). Nucleic acid molecules may be attached through some cleavable group. The cleavage agent (e.g. enzyme or chemical substance) may be delivered after the sandwich assembly.

In yet another embodiment the nucleic acid molecules are held on the original positions in the sample by hybridization and the new solution destabilizes hybridization by changing pH or ionic strength of the solution or decreasing the melting temperature of the duplex like formamide, or the nucleic acid molecules are held on the original positions in the sample by chemical- or enzyme-sensitive binding and said new solution contains a cleavage agent, and wherein either the sample or the target surface or both are permeable for the said solution and during changing of the solution the assembly remains intact. Thus, only the solution is changed but the integrity of the assembly is not changed, i.e. the assembly of the sample and target surface is not disassembled.

If nucleic acid molecules are attached to the sample by hybridization to a complementary sequence, duplexes may be denatured by heating the assembly. Nucleic acid molecules may be covalently attached to the sample through thermolabile bonds like abasic site or formaldehyde linkages. In such cases heating would destroy the binding. Binding may also be organized through enzymatically or chemically cleavable site, where cleavage enzyme or chemical reagent should be thermoactivated. Cleavage agent should then be present in the solution, but during assembling the sandwich it should not act (e.g. to prevent working of an enzyme sandwich may be assembled at low temperature) or should act slowly (e.g. low concentration, inappropriate temperature). In one embodiment light may be applied to release molecules attached to the sample through photocleavable groups. In this case either the nucleic acid on the sample or the target surface or both should be translucent for the light of the required wavelength. Sandwich should be assembled without the activating light.

Under certain conditions it may be necessary to wash the sample or the target surface after incubation. Washing can be performed with known washing buffers, such as PBS or any other washing buffer known to the skilled artisan. Care should be taken not to use washing buffer, which are able to disrupt the bonding between the hybridized nucleic acid molecules and their complementary sequences.

The above disclosed conditions for releasing of nucleic acid molecules from the sample may also be applied in order to release oligonucleotide markers from the target surface when the oligonucleotide markers should diffuse to the surface of the sample, into the sample or into the solution for hybridization.

An “oligonucleotide” as used herein is a short nucleic acid polymer, typically with fifty or fewer bases. Although for the purposes the present invention, the oligonucleotides can have more or less nucleic acids.

Before separating the surfaces, it may be necessary to decrease the temperature close to 0° C. At low temperature hybridization speed becomes low, which prevents attaching of nucleic acid molecules to the wrong places on the target surface when the sandwich-like configuration is disturbed. Optionally, washing of the target surface may be performed at low temperature. Thus, in one embodiment before disassembling the sample and the target surface slowing down formation of new hybrids of nucleic acid molecules is done by decreasing the temperature of the assembly.

In one embodiment a plurality of adapter oligonucleotides is provided. The adapter oligonucleotides are complementary both to the nucleic acid molecules from the sample and to the nucleic acid molecules on the target surface. These adapter oligonucleotides are characterized by at least two regions, wherein one region is at least partially complementary to a nucleic acid on the sample and another region is at least partially complementary to the oligonucleotide markers attached to the target surface. In this embodiment the nucleic acids do not hybridize directly to the at least one type of oligonucleotide markers on the target surface but said hybridization-based binding occurs through adapter oligonucleotides which are complementary both to the nucleic acid molecules from the sample and to the nucleic acid molecules on the target surface.

The general mechanism is a shown in FIG. 2B in comparison to direct hybridization of the nucleic acids to the target surface as shown in FIG. 2A. The use of adapter oligonucleotides allows to use the same target surfaces for hybridization probes with different regions responsible for binding to the target surface.

After the nucleic acids from the sample have been transferred to the target surface enzymatic reactions may be performed with the replica on the target surface, wherein said enzymatic reactions include primer extension, ligation, rolling circle amplification, in situ PCR amplification, bridge PCR amplification, sequencing, restriction (see FIGS. 4 and 5).

In yet another embodiment of the invention the nucleic acid molecules in the sample or the nucleic acid molecules on the target surface contain known sequences, which get inserted in the nucleic acid molecules from the target surface or the nucleic acid molecules from the sample by primer extension or ligation reactions and said known sequences are further used for analysis of replicas, wherein said analysis may be performed on the target surface or in solution.

The term “hybrids” as used herein refers to the direct result of a hybridization of a nucleic acid molecule with an oligonucleotide marker. Furthermore this term includes also all products resulting from further reactions, preferably enzymatic reactions, on such a hybrid such as primer extension or ligation reactions which are performed to integrate the known sequence also in the second strand of the hybrid.

In another embodiment of the invention the known sequences are different between the samples, the target surfaces, replication experiments and serve to distinguish the samples, the target surfaces, and/or replication experiments or (ii) wherein the known sequences are different in different regions of the sample or of the target and serve to determine the position of nucleic acid molecules on the target surface or in the sample.

Oligonucleotide markers on the target surface may contain besides the regions for hybridization-based binding of nucleic acid molecules from the sample, sequences for labeling the transferred nucleic acid molecules. Such sequences get attached to the transferred nucleic acid molecules or their derivatives (extention, ligation products) after replication by ligation or primer extension. In the following analysis of the replicated molecules or their derivatives, for example by sequencing or hybridization, the labeling sequence would reveal to which oligonucleotide a certain replicated molecule was bound.

It is desirable, that nucleic acid molecules do not go off their relative positions in the sample during preparation of the sandwich-like assembly. There are three ways to organize molecular transfer between the sample and the target surface within the inventive methods:
nucleic acid molecules are free or released before preparation of sandwich-like assembly, as described in step c);
nucleic acid molecules are fixed to the sample. Release is started just before preparation of sandwich-like assembly and proceeds after sandwich-like assembly is ready, as described in step c′);
nucleic acid molecules are fixed to the sample and released only after sandwich-like assembly is ready.

Optionally the inventive method comprises after step e) further step e′): e′) providing conditions for slowing down the formation of new hybrids of nucleic acid molecules and marker oligonucleotides. Thereby the formation of new hybrids may be slowed by decreasing of the temperature of the sample or the target surface; by changing the solution between the sample and the target surface wherein the sample or the target surface or both are permeable for a liquid; or by reversing the direction of liquid flow (blotting) or electric field (electrophoresis) to slow diffusion of nucleic acid molecules from sample to the target surface.

While it is important to keep transferred molecules on a new positions for replication, for positional labeling the only requirement is to bind together nucleic acid molecules and correspondent oligonucleotide markers. So, it is possible to release from their positions either (i) only nucleic acid molecules, or (ii) the oligonucleotide markers, or (iii) the nucleic acid molecules and the oligonucleotide markers simultaneously. In the case (i) the nucleic acid molecules from the sample are replicated to the target surface. In the case (ii) the oligonucleotide markers from the target surface are replicated on a sample. In the case (iii) the nucleic acid molecules and the oligonucleotide markers replicas appear within solution in between the sample and the target surface. The relative positions of the nucleic acid molecules (and the oligonucleotide markers) in the solution replica is the same as in the sample (and on a target surface). The only difference from the replicas formed in cases (i) and (ii) is that the solution replica is not attached to the solid surface, but exists temporarily in solution.

The inventive methods disclosed herein are especially useful if samples are provided on which or wherein an arbitrary number of nucleic acid molecules is contained but not in an evenly distributed manner or homogeneously distributed manner or a uniformly distributed manner, because one advantage of the present invention is that the information can be kept and can be obtained where each specific nucleic acid molecule was located in the sample as originally provided. Thus samples unlike fermentation media, waste water or urine are preferably used, wherein the presence or at least the concentration of the nucleic acid molecules which shall be detected is different depending on the location or area of the sample. Thus step a) in all methods disclosed herein could alternatively also read as follows:
a) providing the sample containing nucleic acid molecules located either on the surface of the sample or within the sample, wherein the presence or the concentration of the nucleic acid molecules varies depending of the area of the sample.
   Step a) in all methods disclosed herein could alternatively also read as follows:
a) providing the sample containing nucleic acid molecules located either on the surface of the sample or within the sample, wherein the nucleic acid molecules are unevenly distributed over the surface of the sample or within the sample.

It is further preferred within a method according to the invention that step a) reads as follows:
a) providing the sample containing inhomogeneous distributed nucleic acid molecules located either on the surface of the sample or within the sample.

That the distribution of the nucleic acid molecules within the sample or on the surface of the sample is inhomogeneous refers to samples wherein at least one type of nucleic acid molecule, which means one nucleic acid molecule having a specific sequence is not located in each area of the sample in the same concentration. Alternatively an inhomogeneous distribution occurs if at least one area of the sample differs in its nucleic acid molecules contained (at least one specific nucleic acid molecule is missing or at least one specific nucleic acid molecule is added compared to other areas of the sample).

In one preferred embodiment of the inventive method the hybrids of the nucleic acid molecules and the oligonucleotide markers with known sequences are linked by ligation, by primer extension of oligonucleotide markers on nucleic acids, by primer extension of nucleic acids on oligonucleotide markers by non-covalent association of oligonucleotide markers and nucleic acids, in particular by biotin-streptavidin association, or by chemical association of oligonucleotide markers and nucleic acids.

Labeling sequences may be used for position coding of the transferred nucleic acid molecules. For example, the target surface may be divided into a number of small regions (code regions), oligonucleotide markers in each region containing unique nucleic acid codes—a 4-100 nt nucleic acid sequence. Coding target surface may be used for position coding of transferred nucleic acid molecules: in each code region a different nucleic acid code will be added to the nucleic acid molecules. Adding may be performed by for example ligation, primer extension in appropriate conditions. By adding position-specific codes, information about surface coordinates of nucleic acid molecules is recorded in the sequences of nucleic acid codes. It is then possible to remove the coded replicated nucleic acid molecules from coding surface into solution. In the course of further analysis reading of the codes gives information about original positions of nucleic acid molecules.

In these embodiments the hybridization probes are transferred to a target surface with preformed coded regions—thus, hereinafter named coding surface—and oligonucleotide markers already distributed on the coding surface.

The general procedure is that prior to transfer of the nucleic acids from the sample to the coding surface, so called code regions are created on the coding surface. Thus the coding surface is subdivided in any number of code regions, the number of code regions being dependent on the desired resolution. The code regions can be created physically, by applying e.g. a filter or net on the original surface, wherein each “hole” in this net or filter would represent one code region. It is also possible to use beads with coding oligonucleotides attached to them, wherein each bead would correspond to one coding region. However, it is also possible that the code regions are not created physically but only imaginary code regions are created. This could be realized by e.g. registering the coordinates of each code region on the sample. The coding surface comprises a plurality of coding oligonucleotides attached to the target surface. As long as the coding surface can bind oligonucleotides to its surface, the coding surface can be of any texture. The coding surface consists of code regions in each code region coding oligonucleotides have a different nucleotide code. The more precise localization of transcripts is required, the smaller code regions should be used. The more code regions should be on the coding surface—the longer code regions are required to have a unique code in each code region.

Such coding surface may be prepared for example by spotting nucleic acid codes, by making layer of beads with nucleic acid codes, by synthesizing nucleic acid codes directly on the surface. In one preferred embodiment of the inventive method in step b) two-dimensionally distributed oligonucleotide markers with known sequences are provided as a microarray or as two-dimensionally distributed microbeads, covered with oligonucleotides, preferably with predetermined or random distribution of microarray features or beads.

Even when different oligonucleotides are well isolated from each other on the target surface they may blur during labeling of the nucleic acid molecules. So, the areas correspondent to positions of oligonucleotide markers would overlap with each other. Thus, depending on organization of labeling areas of a sample nucleic acid molecules correspondent to different markers with known sequences may be (i) overlapping or (ii) isolated from each other.

Therefore it is preferred within the method according to the invention that the areas of a sample correspondent to different oligonucleotide markers are overlapping or isolated from each other.

Some diffusion along the target surface may occur during diffusion of the nucleic acid molecules from the sample to the target surface. Diffusion along the target surface leads to distortion of relative positions of molecules after replication, herein also called blurring. One measure to prevent such distortion is minimizing the distance between the sample and the target surface during assembly. The second measure is to subdivide the sample, the target surface or both into isolated regions, wherein the nucleic acid molecules can't cross the borders of said regions during replication. Isolated regions restrict blurring, because diffusion of the nucleic acid molecules along the target surface is restricted by the borders of the isolated regions or areas. Isolated regions may be created by using a mask with isolated holes or by scratching the sample or the target surface. Mask with holes may be located between the sample and the target surface. It is even better, if the mask is pressed into the sample to split the sample in a number of isolated regions. Besides, mask may prevent the direct contact of the sample and the target surface, which is important for prevention of contamination of the target surface because of unspecific binding. Scratching may be used to create borders of the isolated regions by exposing of hydrophobic basis of the sample or of the target surface. The third measure to prevent distortion is to facilitate diffusion into the direction of the target surface by liquid flow (blotting) or by electric field (electrophoresis). For the directional transfer both the sample and the target surface should be permeable for the liquid flow or electric current. Consequently, it is preferred that the sample, the target surface or both are subdivided into isolated regions, wherein the nucleic acid molecules and the oligonucleotide markers can't cross the borders of the regions and wherein the regions are created by using a mask with isolated holes or by scratching the sample or the target surface. Furthermore within another preferred method according to the invention the conditions for diffusion of the nucleic acid molecules or oligonucleotide markers in step e) are facilitated by liquid flow (blotting) or by electric field (electrophoresis).

The transferred nucleic acids would be coded using primer extension reaction: depending on the unique nucleic acid sequence in the coding oligonucleotides, nucleic acids will be extended with a certain unique sequence. The primer extension mix would contain nucleotides and polymerase in an appropriate buffer. Care has to be taken that during primer extension reaction, the nucleic acids do not go off their locations. Therefore, extension should be performed at temperatures below annealing temperature of the nucleic acids.

The result of the extension would be a double-stranded molecule, in which both stands have flanking regions required for sequencing and unique nucleic acid sequence from the coding oligonucleotides, required for revealing the original position on the original surface. The coded extension products can be removed from the double-stranded molecule by different methods. In one embodiment the coding surface is rinsed high-temperature (˜95° C.) solution. At high temperature, the double strands will be denatured and the non-covalently attached stands go into solution. Also high temperature inactivates the enzyme used for primer extension, so that no primer extension is possible in the solution.

In another preferred embodiment of the inventive method one oligonucleotide marker is attached per nucleic acid molecule. It means that a unique code in form of a known sequence should unambiguously correspond to each area of the sample. The number of different codes for <<one code per one area>> labeling is equivalent to the number of distinguishable areas.

In another preferred embodiment of the inventive method two types of oligonucleotide markers are attached to each nucleic acid molecule: one to the 3′ end and another one to the 5′ end. This approach requires association of the nucleic acid molecules with two types of marker oligonucleotides, wherein each type has a different known sequence. The advantage is a possibility to use smaller number of oligonucleotide markers for coding. It is necessary to have one million of individual oligonucleotides for <<one code per one area>> coding of one million different positions. But the number of oligonucleotides decreases to 2000 for combinatorial labeling with two types of markers: 1000×1000=10⁶. The possible realization of combinatorial labeling is to set up a Cartesian coordinate system on the surface and use one type of markers for coding of “X”-coordinates, and another type of markers for coding of “Y”-coordinates.

In another embodiment, the coding oligonucleotides on the coding surface would further comprise a cleavable group. Due to this cleavable group, the whole double strand can be removed from the coding surface after destroying the cleavable group. The double strand may be further amplified and then sequenced.

It should be taken into consideration, that during transfer of nucleic acid molecules to the coding surface and during adding of nucleic acid codes to the nucleic acid molecules, nucleic acid codes should stay within the coding regions. Depending on the way of attachment of coded replicated nucleic acid molecules to the coding surface, nucleic acid molecules may be released independently from non-used nucleic acid codes or together with them. For example, when coded nucleic acid molecules are attached to the coding surface by hybridization, and nucleic acid codes are covalently attached, nucleic acid molecules may be released from the surface by denaturizing conditions, and nucleic acid codes will remain on the surface. When both nucleic acid codes and coded replicated nucleic acid molecules are attached to the coding surface in the same way, they will be released together. In the latter case nucleic acid codes either remain in the mixture with coded nucleic acid molecules if they do not interfere with further operations, or they would be removed, for example by size selection.

The present invention is also directed to a coding surface with a plurality of coding regions, wherein the coding surface is covered with a plurality of coding oligonucleotides, wherein the coding oligonucleotides are characterized by a 3′ part common to all coding oligonucleotides, and an individual nucleotide sequence of 4-100 nucleotides, characterized in that each coding region is covered only with coding oligonucleotides with the same individual nucleotide sequence of 4-100 nucleotides.

In another preferred embodiment of the method according to the invention the analysis of the hybrids of the nucleic acid molecules and the oligonucleotide markers is performed by sequencing. Sequencing is a convenient method, because in any case sequencing is inevitable for decoding of marker oligonucleotides. It is possible to prepare Next Generation Sequencing (NGS) library in such a way, that a separate read would be used for sequencing of code (known sequence of the oligonucleotide marker) and the nucleic acid molecule. Another possibility is to prepare NGS-library in such a way, that a part of the read would correspond to the code and another part to nucleic acid. This means that in order to get the information about the spatial distribution at least the part with the known sequence of the oligonucleotide marker has to be sequenced. This can be enough, for example when only one known sequence of interest can hybridize to the oligonucleotide marker and the user is interested in the distribution of this specific nucleic acid molecule. Furthermore the nucleic acid molecules may be analyzed by other methods like restriction enzymes. Nevertheless especially when the nucleic acid molecules from the sample are not

Specification known or not completely known it is preferred to sequence also the nucleic acid molecules originated from the sample.

Currently sequencing is used in a number of applications: transcriptome analysis, resequencing, genotyping, epigenetic studies, analysis of microbiomes and biological diversity, etc. In combination with identification of original positions of nucleic acids described in the current invention sequencing is especially useful for expression profiling, locus specific sequencing, or analysis of methylation status of particular loci in tissue sections. When the analysis of the hybrids is performed by sequencing the methods of the present invention are suitable for expression profiling, locus specific sequencing, or analysis of methylation status of particular loci in tissue sections.

Expression profiling of tissue sections allows analyzing expression pattern of a number of genes in parallel. The number of sequences correspondent to the gene is proportional to the expression level. Positions correspond to distribution of gene-specific mRNA in tissue section.

Locus specific sequencing of tissue sections allows to recognize somatic mutations and to distinguish subpopulations of tumor cells. It may be especially useful for screening of state of oncogenes in individual tumor cells.

Methylation status is important for a number of molecular processes from gene expression to cellular differentiation. Analysis of methylation status in tissue sections is useful for studies in this field and for revealing of molecular mechanisms of different pathologies at a single-cell resolution level.

Normally, tissue sections contain heterogeneous population of cells. Sequencing with position identification permits to characterize cells individually. It is important for functional analysis of complex tissues and for revealing of dangerous subpopulations of cells in heterogeneous tumors.

In the following some preferred methods are described in more detail.

Currently, transcripts in tissue sections are analyzed by in situ hybridization. Main restriction of this approach is the limited number of transcripts which it is possible to analyze simultaneously. The reason is that it is impossible to select considerable number of distinguishable labels for hybridization probes. Transcripts in tissue sections may be analyzed by sequencing and ex situ hybridization as follows:

In the second generation sequencing (SGS) platforms sequencing is performed on the surface of a special flowcell for millions of templates in parallel. 2D flowcell surface is similar to the slide with tissue section. Sequencing cannot be performed directly in the tissue section. However using a method of the invention it is possible to transfer the transcripts (hybridization probes, primer extension products) from tissue section to the surface of the sequencing flowcell preserving the distribution pattern.

The method may be conducted according the following flow chart:

Hybridization probes should have the structure as shown in FIG. 6A. Middle parts of probes are for hybridization to transcripts in tissue section. Flanking regions a and b are common for all probes and are required both for hybridization and sequencing on the SGS flowcell surface.

Hybridization probes may be selected to target from single to thousands of transcripts. They may be synthesized artificially or prepared from a sequencing library. To prevent unspecific hybridization of common parts of the hybridization probes in tissue section it is possible to reversibly block them with complementary oligonucleotides. These oligonucleotides should be removed before transfer of hybridization probes to the SGS flowcell surface.

Tissue section slide and SGS surface would be brought into tight contact, possibly with a net in between (see FIG. 3). The distance between surfaces (or the mesh size if a net is used) should be smaller than acceptable blurring of the distribution pattern. A net would also prevent direct contact of the tissue section and SGS surface.

Surface assembly would be done at room or lower temperature, so that the hybridization probes do not go off the surface. Detaching probes from the tissue section and attaching to the SGS surface would be regulated by the temperature. First, the temperature of the sandwich would be raised up to denaturize the hybridization probes. Then the temperature would be decreased to allow the common regions of hybridization probes annealing to the oligos immobilized on the SGS surface. In these conditions hybridization probes may hybridize back to the transcripts in tissue sections. However transcripts in the tissue section are few in comparison to oligos on the target surface, so probability to hybridize to the target surface is much higher than back to the tissue section.

The time of denaturation would be selected to allow enough probes to denature and move into solution between the surfaces. The time of hybridization should be adjusted so that enough but not too many probes are transferred to provide a necessary density of sequencing templates and so that probes do not diffuse too far away. Before separating the surfaces, the temperature would preferably be decreased close to 0° C. At low temperature hybridization speed becomes low, which prevents attaching of probes to the wrong places on SGS surface when sandwich is disturbed. Washing of the unhybridized probes from the SGS flowcell surface would be also performed at low temperature.

Amplification of the transferred probes on the SGS flowcell surface and further sequencing would be performed according to the known sequencing procedures (FIG. 6). SGS would determine two parameters for each probe: (i) its partial or complete nucleotide sequence and (ii) position on the slide surface. Nucleotide sequence will identify which particular transcript was a target for a probe. Position of a probe on a flowcell will be set into correspondence with the position on the tissue section.

An alternative to SGS analysis is the analysis of transcripts in tissue sections by single molecule sequencing transfer of transcripts distribution pattern to the pattern of sequencing templates (FIG. 7).

The procedure looks the same as described before, with the difference in sequencing approach: molecules transferred from the tissue section are sequenced directly by single-molecule sequencing approach, where transferred molecules are sequenced directly on the target surface with capturing oligonucleotides initializing the primer extension. Since no amplification on the target surface is required, only one type of oligonucleotides can be present on the sequencing surface for capturing sequencing templates by hybridization. This approach may be realized using single-molecule sequencing approach like for example that of Helicos. Single molecules sequencing allows for a higher density of sequencing templates.

A further alternative to SGS analysis is analysis of transcripts in tissue sections by ex situ hybridization. The procedure looks the same as described before but amplification of the transferred nucleic acid molecules on the target surface and removing of one strand. Then instead of sequencing, target surface is used for hybridisation with probes of interest. So, this is basically in situ hybridization but with targets transferred to another surface and amplified.

In situ amplification results in ˜1000 copies of transferred molecule. This allows increasing hybridization signal and thus sensitivity of transcripts analysis. Another advantage of this approach is that it makes possible to use same replica for several hybridizations with different probes without increase of background. Target molecules are covalently attached to the surface, so it is possible to use stringent conditions to wash off probes from previous hybridization. This increases the throughput of analysis in comparison to in situ hybridization.

Another preferred embodiment refers to marking positions of transcripts in tissue section by nucleic acid codes using a coding surface and subsequent analysis by SGS sequencing

This method allows to transfer transcripts (or corresponding to transcripts hybridization probes, primer extension products) from tissue section into solution and thereby preserving information about the distribution pattern. Molecules in the solution may be further processed according to standard sequencing protocols for sample preparation. Loading of sequencing flowcell would be performed as for standard sequencing library, so loading density will be even over the flowcell surface and adjustable. Having sequencing templates in the solution would also allow to use any SGS platform and thus be independent from the SGS surface.

The possible procedure of this preferred method is:

Middle parts of probes are for hybridization to transcripts in tissue section. Flanking regions are common for all probes and are required for hybridization to the coding surface (hybr. region) and sequencing on the SGS flowcell surface (seq. region 1).

To prevent unspecific hybridization of common parts of the hybridization probes in tissue section it is possible to reversibly block them with complementary oligonucleotides. These oligonucleotides should be removed before transfer of hybridization probes to the coding surface.

The coding surface is covered with covalently attached coding oligonucleotides. The 3′ part, which is complementary to the hybridization region of the hybridization probes, is followed by code region. 5′ part is required for further sequencing on the SGS flowcell (seq. region 2). Coding oligo may be detached from the surface using a cleavage site. Cleavage site may be organised for example by a chemically, thermally or enzymatically destroyable nucleotide.

Coding surface consists of coding regions, in each region coding oligos have a different code part. The more precise localisation of transcripts is required, the smaller coding regions should be used. The more coding regions should be on the surface—the longer code region is required to have a unique code in each region.

Hybridized probes would be transferred to a coding surface as described before. Attachment to the coding surface would be realized by hybridisation of the hybridization region of the hybridization probes to the complementary 3′ part of the oligos on the coding surface. The result of the transfer would be a coding surface with hybridization probes attached to it in a mirror-distribution relative to the distribution of corresponding transcripts in tissues section.

Transferred hybridization probes would be coded using primer extension reaction: depending on the coding region, hybridization probe will be extended with a certain code sequence. Primer extension mix would contain nucleotides and polymerase in an appropriate buffer. Mix would be pipetted over the surface using for example HybriWell chambers from Grace Biolabs. It is important that during primer extension reaction, hybridization probes do not go off their locations. Extension should therefore be performed at temperatures below annealing temperature of hybridization region.

The result of the extension would be double-stranded molecules, in which both strands have flanking regions required for sequencing and code regions, required for revealing molecules position. Coded molecules can be removed from the slide and combined in the solution. This may be performed in two ways.

Variant 1. Coding surface would be rinsed in high-temperature (˜95° C.) solution. At high temperature, duplexes will be denatured and non-covalently attached strands will go into solution. Also high temperature would inactivate the enzyme used for primer extension, so that no primer extension would be possible in the solution (which may cause chimeric molecules formation). Single-stranded sequencing templates have common flanking regions required for SGS and may be further amplified or used directly for clonal amplification.

Variant 2. Duplexes would be removed from the coding surface after destroying of the cleavable group. Together with duplexes, non-extended coding oligos will also be removed from the coding surface, and may cause extension in solution, which may lead to wrong coding and formation of chimeric molecules. It is therefore necessary to pay attention that polymerase present in primer extention mix is washed away from the surface or inactivated prior to combining the duplexes in solution. Double-stranded sequencing templates may be further amplified or used directly for clonal amplification.

Further stages—amplification of the molecules, clonal amplification and sequencing would be performed according to the known SGS procedures (SOLiD platform from ABI; GA and HiSeq from Illumina).

SGS would determine two sequences for each sequencing template: (i) partial or complete transcript-specific sequence and (ii) sequence of the code. Code sequence will be set into correspondence with the distribution scheme of position coding primers on the tissue section slide, and reveal the initial position of the transcript in the tissue section.

Further preferred embodiment refers to marking positions of nucleic acids in tissue section with a sequenced SOLiD flowcell as a coding surface and subsequent analysis by Second Generation Sequencing (SGS), FIGS. 8 and 9.

An already sequenced SOLiD flowcell is used as the coding surface. Clonally amplified sequencing templates are attached to the beads. After sequencing, position of each bead and sequence of molecules attached to it are known. Thus, sequences may serve as codes for hybridization probes transferred from tissue section slide.

Hybridization probes would have middle parts for hybridization to transcripts in tissue section. Flanking regions are common for all probes and are required for hybridization to the coding surface (hybridization region) and sequencing on the

Illumina platform (illumination region 1). Hybridization region may hybridize to the common 3′ region (P2) of SOLiD sequencing templates. To prevent unspecific hybridization of common parts of the hybridization probes in tissue section it is possible to reversibly block them with complementary oligonucleotides. These oligonucleotides should be removed before transfer of hybridization probes to the coding surface.

Coding surface is a sequenced SOLiD flowcell: glass slide covered with beads. Each bead is a different code region. Unique middle parts of sequencing templates serve as codes. Hybridized probes would be transferred to the coding surface as described before. Attachment to the sequencing templates would be realized by hybridization of the hybridization region of the hybridization probes to the complementary P2 regions. The result of the transfer would be beads with hybridization probes attached to them.

Transferred hybridization probes would be coded using primer extension reaction: depending on the bead to which it is attached, hybridization probe will be extended with a certain code sequence. Primer extension mix would contain nucleotides and polymerase in an appropriate buffer. Mix would be pipetted over the surface using for example HybriWell chambers from Grace Biolabs. It is important that during primer extension reaction, hybridization probes do not go off their locations. Extension should therefore be performed at temperatures below annealing temperature of hybridization region. Sequencing templates would not be extended because in the course of the SOLiD sequencing protocol they are 3′ end blocked.

The result of the extension would be a hybridization probe to which the sequence of a SOLiD sequencing template is added, and which has a P1 sequence on 3′end. Coded molecules may be washed off the beads in denaturizing conditions and combined in solution. Single stranded coded molecules would be amplified to introduce illumination region 2 next to P1 part of the molecule. Result of amplification would be double-stranded molecules flanked with Illumina-platform specific illumination regions 1 and 2, which may be further amplified or used directly for clonal amplification and sequencing on the Illumina platform.

Illumina sequencing would determine two sequences for each sequencing template: (i) partial or complete transcript-specific sequence and (ii) sequence of the code. Code sequences will reveal the position of corresponding beads on the SOLiD flowcell and thus the position of original transcripts in the tissue section.

In the previous preferred methods described the aim was to reveal position of the nucleic acid molecules distributed within tissue section. For analysis of a panel of samples with 2D distributed nucleic acid molecules (e.g. cell arrays, tissue arrays) it may be necessary to reveal from which sample nucleic acid molecules originate. Previously described procedures work for these applications, too. If coding is used to mark nucleic acid molecules from a single sample, size of coding regions on the coding surface may be comparable to the size of a sample.

### DESCRIPTION OF FIGURES

FIG. 1: Scheme of positional coding involving the target surface. Hybridization probes are transferred to a coding surface

FIG. 2: Hybridization-based binding of nucleic acids to the target surface. (A) Hybridization directly to the oligonucleotides attached to the target surface. (B) Binding to the target surface by hybridization through adapter oligonucleotides.

FIG. 3: Replication of 2D distributed nucleic acid molecules to an oligonucleotide marker coated target surface.

FIG. 4: Examples of enzymatic reactions which may be performed with replicated nucleic acid molecules (hybridization probes) on the target surface.
(A) Ligation. (B) Primer extension.
Subsequently, replicated nucleic acid molecules can be sequenced on the target surface, using the oligonucleotides on the target surface to start sequencing-by-synthesis or replicated nucleic acid molecules may be amplified, for example by rolling circle amplification (RCA), in situ PCR or bridge PCR.

FIG. 5: Scheme of rolling circle amplification of the replicated nucleic acid molecules (hybridization probe). Replicated nucleic acid molecule is circularised and amplified using oligonucleotides on the target surface first as a template for ligation and then as a primer for amplification.

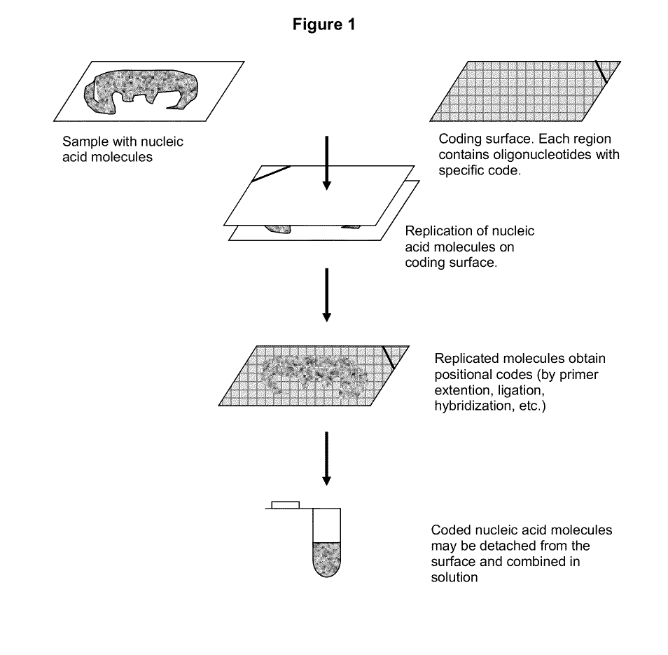

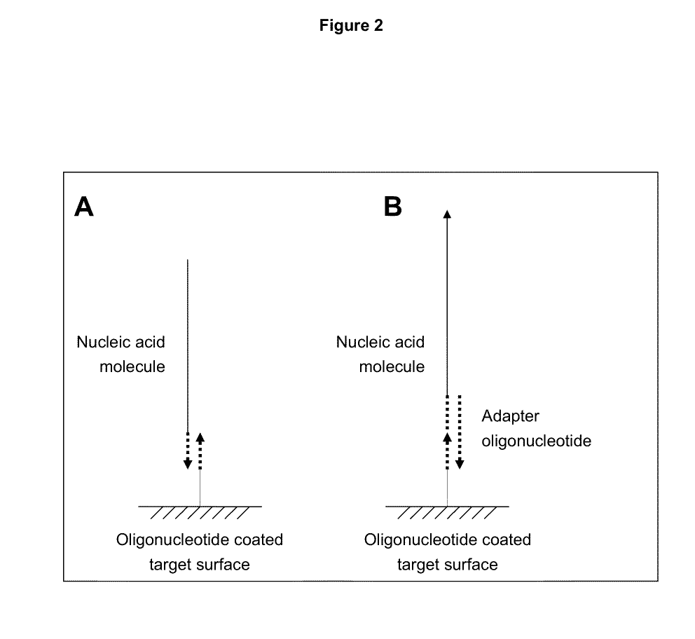

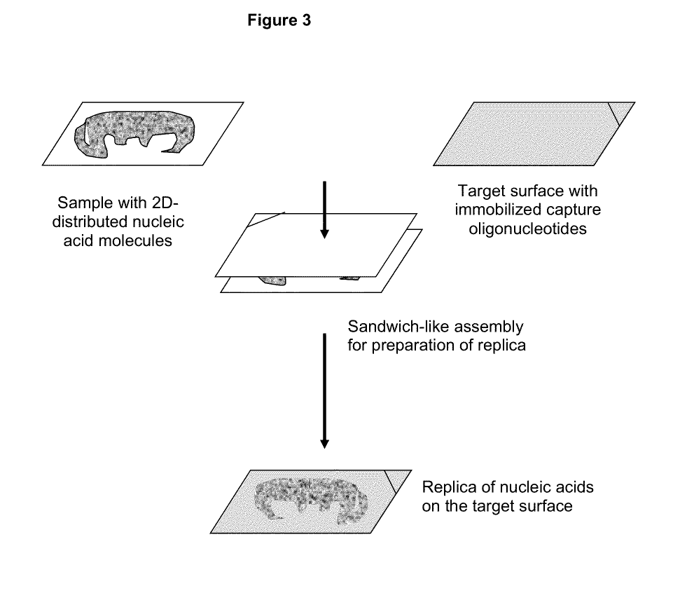

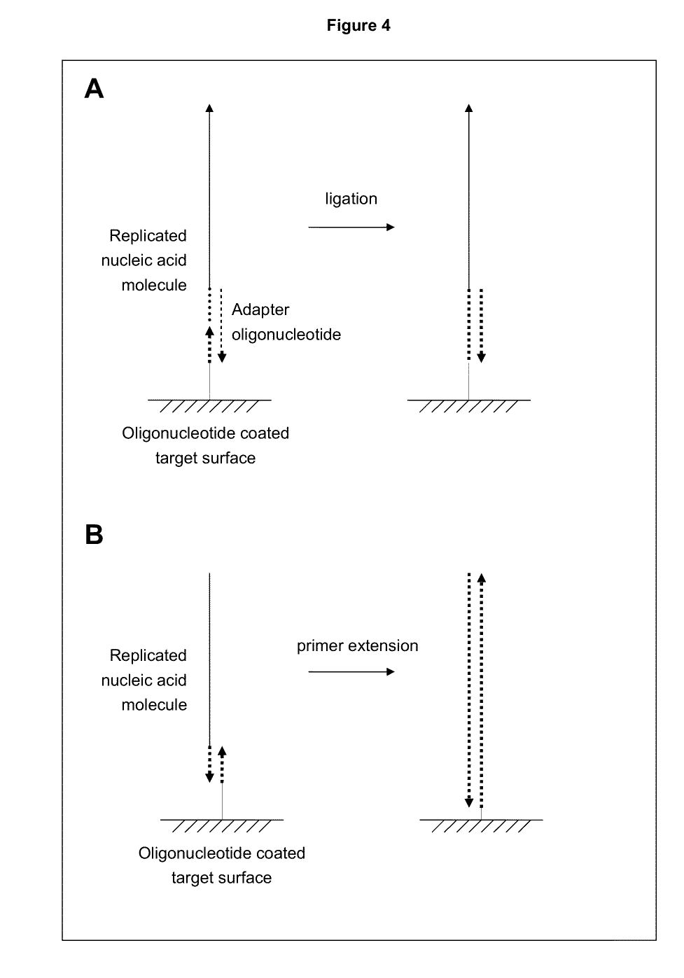

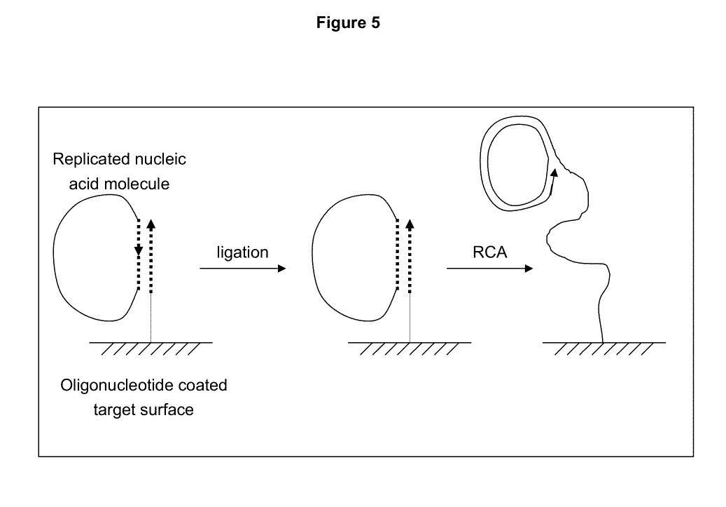

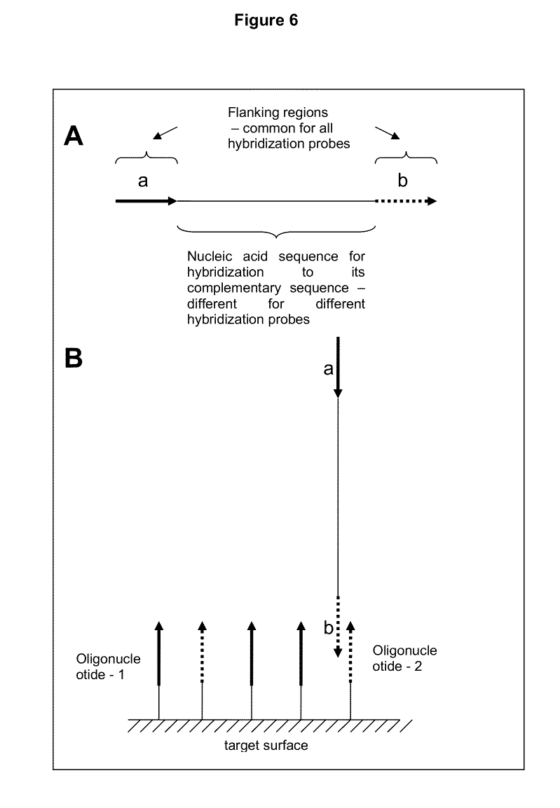

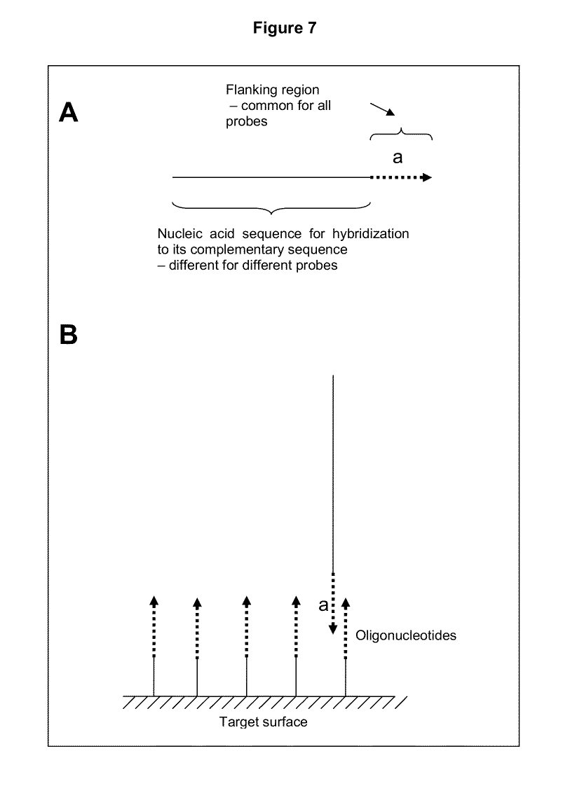

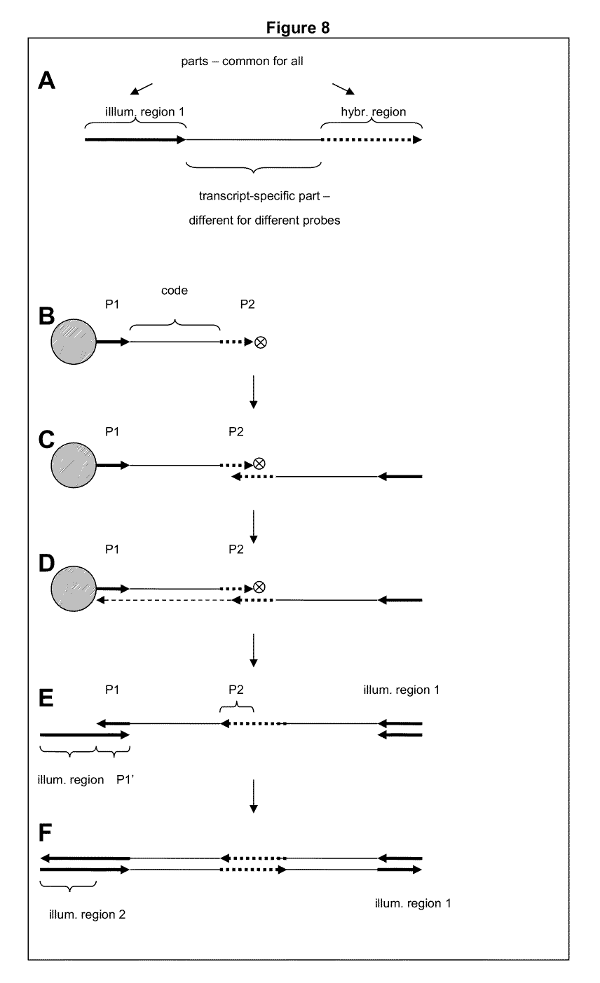

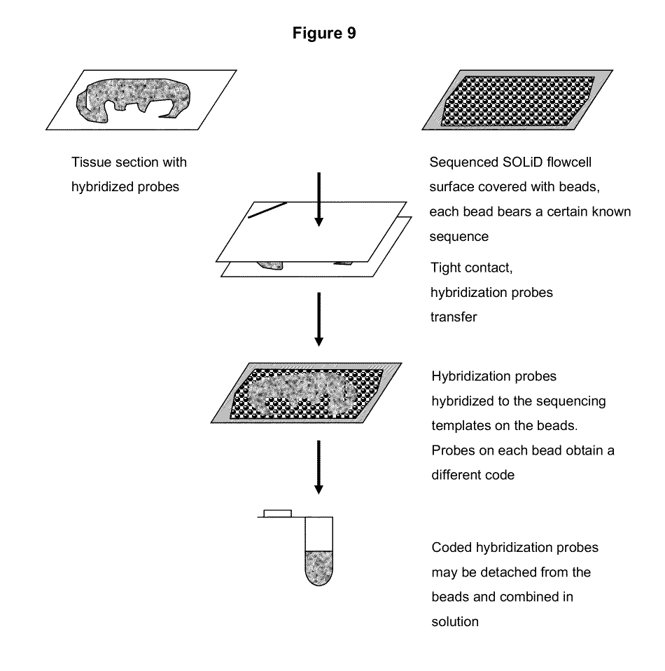

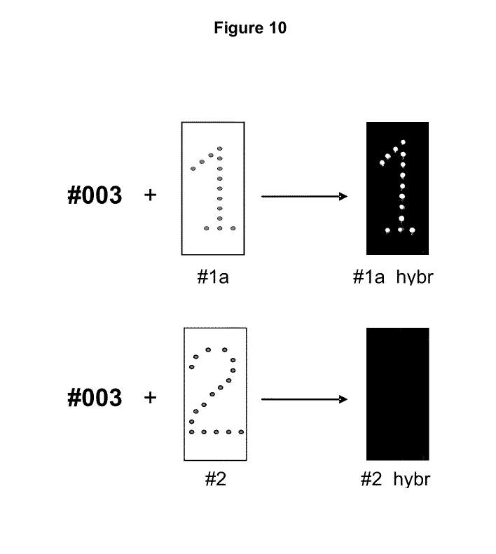

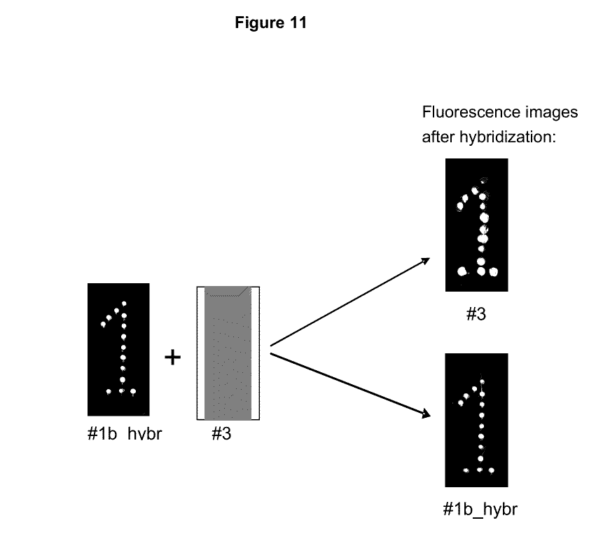

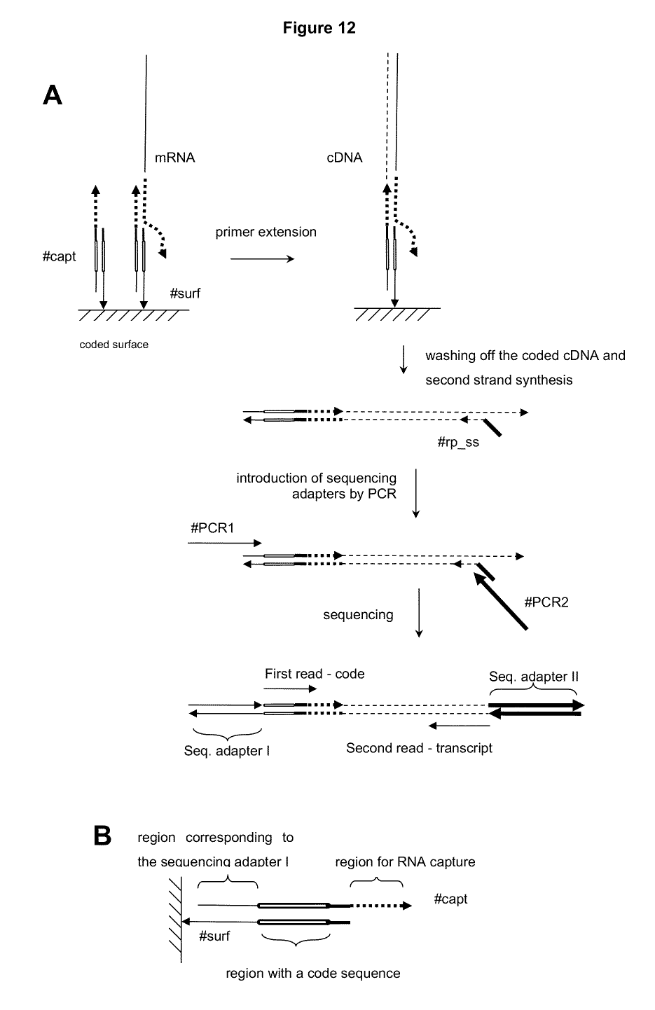

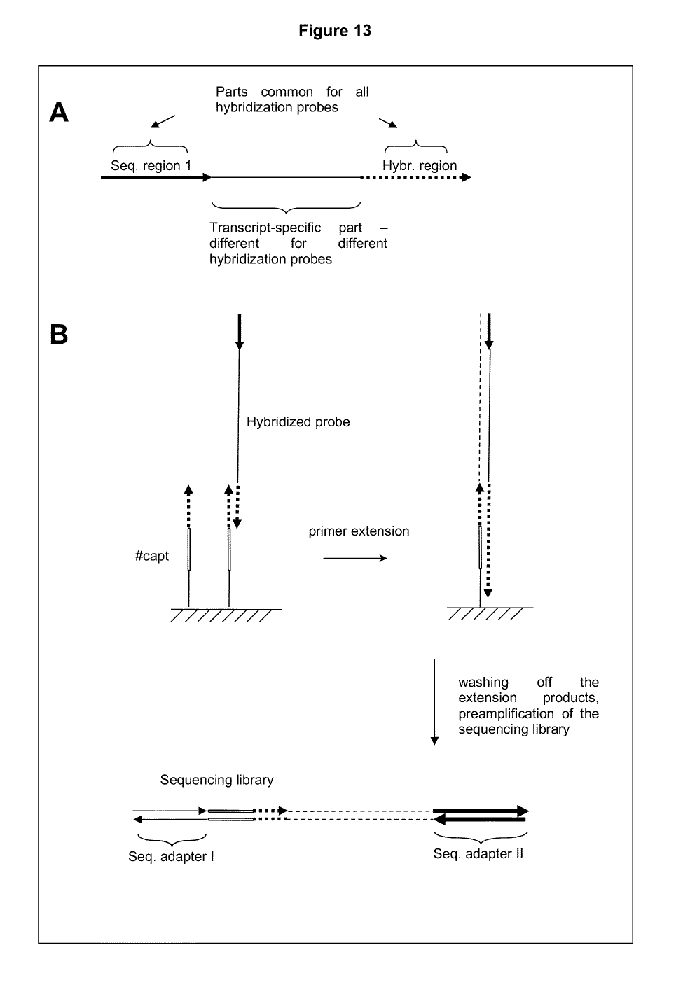

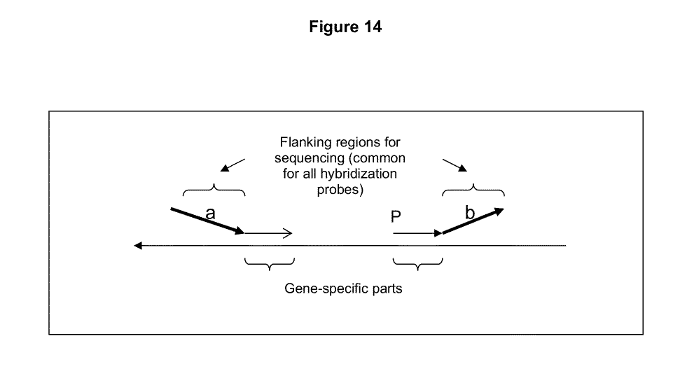

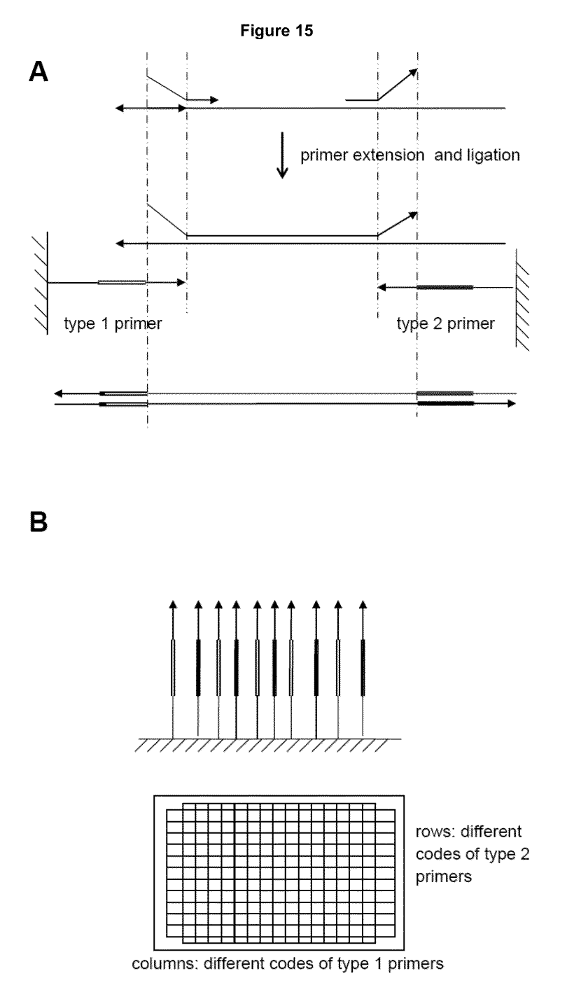

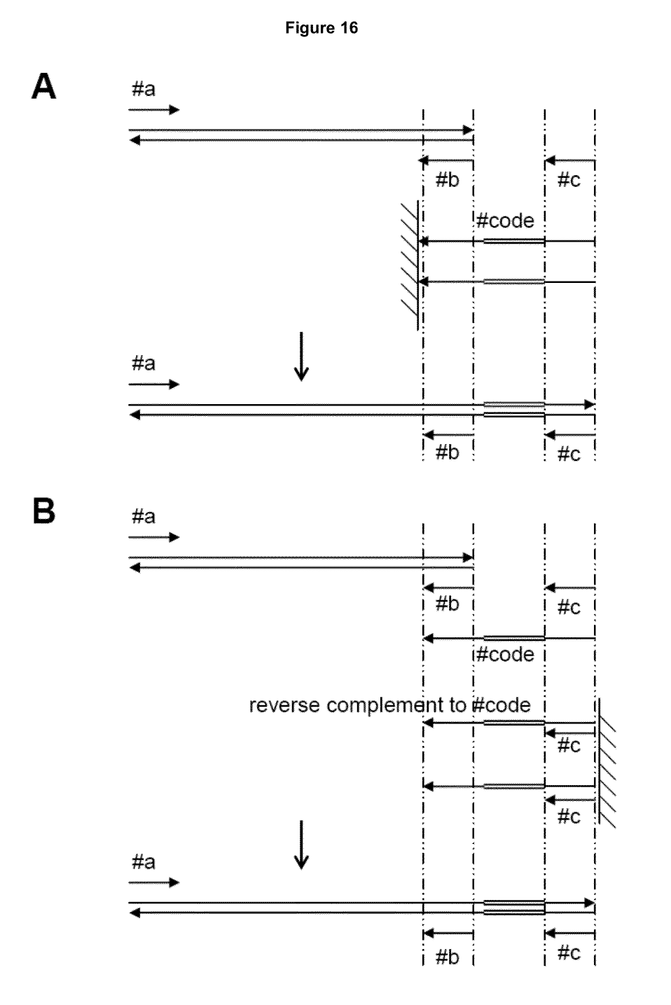

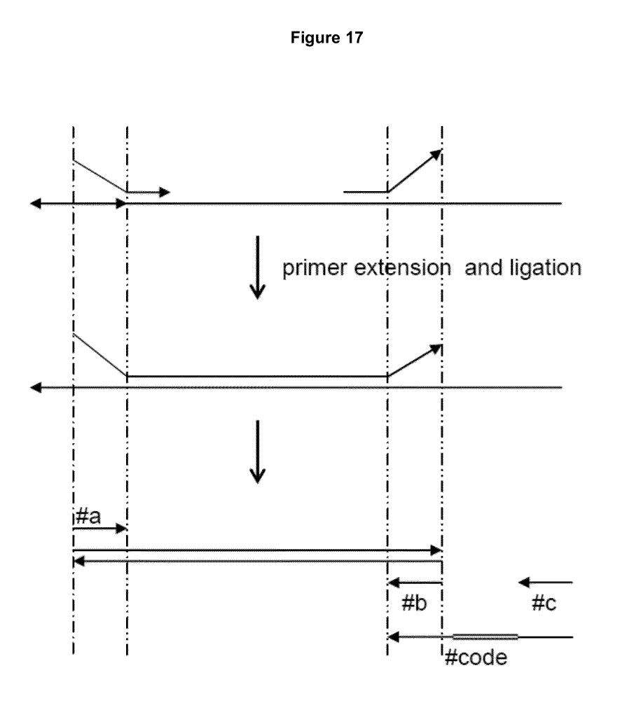

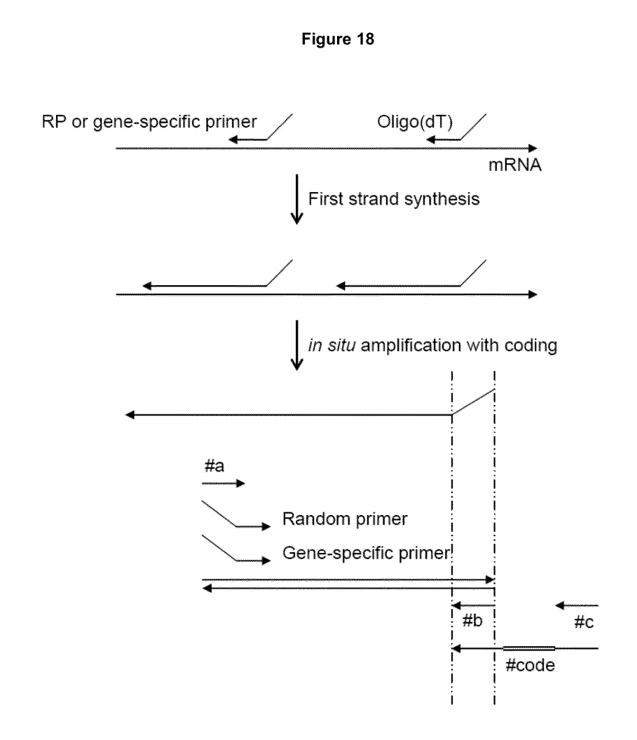

FIG. 6: (A) Structure of hybridization probe with two flanking regions suitable both for (i) hybridization to nucleic acids on sample surface and for (ii) second generation sequencing.
(B) Scheme of the probe hybridization to the target surface. The target surface is covered with attached oligonucleotides-1 and -2. Oligonucleotides-2 hybridizes with region b of the hybridization probe, providing attachment to the target surface. Oligonucleotides-1 hybridizes with sequence complementary to region a of the hybridization probe, making possible the on-surface amplification and second generation sequencing.

FIG. 7: (A) Structure of hybridization probe with one flanking region suitable both for (i) hybridization to nucleic acids on sample surface and for (ii) second generation sequencing. (B) Scheme of the probe hybridization to the target surface. The target surface is covered with attached oligonucleotides. The oligonucleotides hybridize with region “a” of the hybridization probe, providing attachment to the target surface. Second generation sequencing can be performed without amplification according to true single molecule sequencing tSMS™ (Helicos).

FIG. 8: (A) Structure of hybridization probe suitable for (i) hybridization to transcripts in tissue section, (ii) hybridization to the SOLiD P1 region of sequencing templates and having ilium. region 1 necessary for Illumina SGS. (B) Structure if the SOLiD sequencing template attached to the bead. (C-F) Scheme of adding a code to hybridization probes using primer extension. Hybridization probes transferred from tissue section slide hybridize to the P2 region (C). Hybridization probes are extended (D). Internal sequence of the sequencing template which marks the position of the bead on the SOLiD flowcell is now added to the hybridization probe sequence, thus marking the position of the transfer nd of original transcript in tissue section. To introduce ilium. region 2 necessary for Illumina SGS, coded hybridization probes are PCR amplified. One of PCR primers has a P1-complementary 3′ end and ilium. region 2 5′ tail; another primer correspond to ilium. region 1 (E). Resulting double-stranded molecules are suitable for Illumina SGS.

FIG. 9: Position coding involving sequenced SOLiD flowcell as a coding surface. Hybridization probes are transferred to a coding surface covered with beads. Each bead is characterised by a specific coding sequence.

FIG. 10: Cy3-labeled oligonucleotides #003 were hybridized to the slides #1 a (with oligonucleotides #001 deposited to form figure “1”) and to the slide #2 (with oligonucleotides #002 deposited to form figure “2”).

FIG. 11: Transfer of Cy3-labeled oligonucleotide #003 hybridized to the slide #1b_hybr to the slide #3. The surface of the target slide #3 is covered with covalently immobilised oligonucleotides #001 (grey area), which is complementary to #003.

FIG. 12: (A) Scheme of spatially resolved transcriptome sequencing. (B) Structure of oligonucleotides on the coding microarray. Oligonucleotides #surf are chemically synthesized directly on the microarray and their 3′ ends are attached to the glass. 3′ regions of oligonucleotides #capt are single stranded and are used to capture mRNA, middle parts contain codes, corresponding to certain features of microarray and 5′ regions are required for sequencing.

FIG. 13: (A) Structure of hybridization probe suitable for (i) in situ hybridization and for (ii) hybridization to the coding surface. 5′ region of the probe corresponds to the sequencing adapter.
(B) Scheme of spatially resolved locus-specific transcriptome sequencing.

FIG. 14: Structure of probes for locus specific sequencing. After primer extension and ligation the internal part becomes a copy of specific gene locus. 5′ end region of the ligated probe is a sequencing adapter and 3′ end region is complementary to oligonucleotides on the target surface.

FIG. 15: Positional coding with replication of two-dimensionally distributed oligonucleotide markers.
(A) Genotyping by extansion and ligation followed by bridge amplification with two types of coded primers.
(B) Positions of oligonucleotide markers on the microarray. Type 1 primers with a same code form columns; type 2 primers with a same code form rows. In each square of microarray there is a unique combination of two codes.

FIG. 16: Scheme of the in situ PCR with spatial coding (in situ SC-PCR). Oligonucleotides attached to glass slide on (A) and (B) are reverse complement to each other.
(A) Oligonucleotides #code are attached to the glass slide
(B) Oligonucleotides #code are synthesized in situ.

FIG. 17: In situ SC-PCR for genotyping.

FIG. 18: In situ SC-PCR for amplification of expression profiles

### EXAMPLES

### Example 1

### Replication of Oligonucleotides Attached to the Original Surface by Hybridization

### Consumables

Epoxy-modified glass slides: Nexterion Epoxysilane 2-D surface Slide E kit (Schott, #1066643)
Hybridization chambers: Secure Seal (Grace bio-labs, #SA500)
Oligonucleotides:
   SH-modified oligonucleotides for immobilization on the epoxy slides:

#001

(SEQ ID NO: 1)

5′ SH-TTTTTTTTTTAATGATACGGCGACCACCGA 3′
#002

(SEQ ID NO: 2)

5′ SH-TTTTTTTTTTCAAGCAGAAGACGGCATACGA 3′

The unique sequences correspond to the sequences of oligonucleotides immobilized on the Illumina sequencing flowcells;
Cy3-labeled fluorescent hybridization probe:

#003

(SEQ ID NO: 3)

5′ Cy3-AGAGTGTAGATCTCGGTGGTCGCCGTATCATT 3′

Partly complementary to oligonucleotides #001, complementary sequence is underlined.

### Slides Prepared

SH-modified oligonucleotide #001 was immobilized on five epoxy slides: on three slides—in a recognizable pattern and on the other two—over the whole surface. SH-modified oligonucleotide #002 was immobilized on three epoxy slides: on one slide—in a recognizable pattern and on the other two—over the whole surface.
1. 40 μM SH-modified oligonucleotides were diluted to 20 μM by adding the equal volume of the 2× Nexterion Spot solution.
2. Oligonucleotide solutions were deposited on slides:
   Slides #1a, #1b and #1c: 1 μl drops of oligonucleotide #001 were deposited to form a figure “1” (see FIG. 10, #1a):
   Slide #2: 1 μl drops of oligonucleotide #002 were deposited to form a figure “2” (see FIG. 10, #2)
   Slides #3& 4: were laid upon each other with 3 mm wide and 0.2 mm thick spacers along the long sides and oligonucleotide #001 was pipetted to fill the space between the two slides;
   Slides #5& 6: were laid upon each other with 3 mm wide and 0.2 mm thick spacers along the long sides and oligonucleotide #002 was pipette to fill the space between the two slides.

Further all slides (#1a, b, c-6) were handled the same way.
3. Slides with deposited oligonucleotides were incubated in a humidity chamber at room temperature for 30 min to ensure quantitative immobilization.
4. Slides were washed at room temperature:
   for 5 min in 0.1% Triton® X-100;
   two times for 2 min in 1 mM HCl solution;
   for 10 min in 100 mM KCl solution;
   for 1 min in bidistilled water.
5. Blocking was performed:
   incubating for 15 min in 1× Nexterion Blocking Solution at 50° C.;
   rinsing for 1 min in bidistilled water at room temperature.
6. Slides with immobilized oligonucleotides were dried under nitrogen stream and stored in dry atmosphere in an excicator.

### Hybridization of Cy3-Labeled Oligonucleotides to the Slides #1a, b, c and #2.

1. Cy3-labeled oligonucleotides #003 solution was prepared: 10 nM oligonucleotide in 90% Nexterion Hybridization buffer.
2. Hybridization chambers were placed over the areas with spots on slides #1a, #1b, #1c and #2, the labelled oligonucleotides solution was added to the chamber.
3. Slides were incubated for 1 hour at 42° C. in the PCR machine with glass slides adapter.
4. Hybridization chambers were removed and slides were washed at room temperature:
   for 10 min in (2×SSC, 0.2% SDS);
   for 10min in 2×SSC;
   for 10min in 0.2×SSC.
5. Slides #1b and #1c with hybridized Cy-3 labeled oligonucleotide (#1b_hybr and #1c_hybr) were left in 0.2×SSC at room temperature for ˜1 hour, till the transfer experiment was performed.

6. Slides #1 a and #2 with hybridized Cy-3 labeled oligonucleotide (#1 a_hybr and #2_hybr) were dried under nitrogen stream and scanned on the Affymetrix 428 Array Scanner.

On slide #1a_hybr a fluorescent pattern of the figure “1” was obtained (see FIG. 10). No fluorescent signal was observed on the slide #2_hybr.

Transfer of the Cy-Labeled Oligonucleotide #003 Hybridized to Slides #1b_hybr and #1c_hybr to the Slides #3 and #5

Cy3-labeled oligonucleotides #003 hybridized to the slide #1b_hybr was transferred to the slide #3 covered with oligonucleotide #001, complementary to #003. Cy-3 labeled oligonucleotide #003 hybridized to the slide #1c_hybr was transferred to the slide #5 covered with oligonucleotide #002, not complementary to #003.
1. ˜25 μl of Nexterion Hybridization buffer was pipetted on the oligonucleotide covered surfaces of slides #3 and #5, to which the Cy-3 labeled oligonucleotide had to be transferred.
2. Slides #1b_hybr and #1c_hybr with the hybridized fluorescent oligonucleotide #003 were placed over the drop of Nexterion Hybridization buffer on slides #3 and #5 respectively.
3. The sandwiches of slides #1b_hybr/#3 and #1c_hybr/#5 were placed in separate plastic bags.
4. The slides in both sandwiches were pressed tightly to each other with paper clips to let the hybridization buffer squeeze out into the bag.
5. Bags with slide sandwiches were placed in a beaker with boiling water for 3min.
6. Bags were transferred to a beaker with 42° C. water for 15 min.
7. Bags were transferred to room temperature; sandwiches were taken out and disassembled. All slides were washed, blocked, dried out and scanned as described in the “Hybridization” section.

On slide #3a mirror replica of the fluorescent pattern of the figure “1” from slide #1 b_hybr was obtained. Thus, the Cy-3 labeled oligonucleotide #003 hybridized to the slide #1 b_hybr has been transferred to the surface of slide #3 (see FIG. 11). No transfer of oligonucleotide #003 from slide #1 c_hybr to the slide #5 was observed.

### Example 2

### Spatially Resolved Transcriptome Sequencing

Example demonstrates positional labelling of mRNA from tissue section and subsequent spatially resolved transcriptome analysis by sequencing. The scheme of the experiment is shown on FIG. 12.

Spatially resolved transcriptome sequencing includes five stages. On the first stage mRNA molecules from tissue section are replicated to the microarray and get captured by hybridization to single-stranded oligo(dT) regions of oligonucleotide markers. Microarray contains 10⁶ individual features and potentially is capable to provide about 40 μm resolution.

Positionally coded first strand cDNA molecules are synthesised on the second stage. First strand synthesis is performed on the surface of the microarray. Oligonucleotide markers are extended along the hybridized mRNA molecules. Obtained cDNA molecules have the first sequencing adapter adjacent to the code on 5′ ends and transcript-specific part on the 3′ ends.

Other stages (3-5) of spatially resolved transcriptome sequencing are performed using kits for standard molecular biology protocols.

On the third stage coded cDNA molecules are washed out from the microarray. Sequencing library is generated by second-strand synthesis from random primers combined with second sequencing adaptors. After size selection and preamplification the library is ready for sequencing.

Sequencing is performed in paired-end mode. The first read identifies codes, the second—transcripts.

On the final stage sequencing reads correspondent to individual genes are grouped together and each group is used for preparation of the expression maps of individual gene. Each sequencing read was presented as a point on a picture. Position of the point was selected according to the code associated with the sequencing read.

Replication of mRNA from a Tissue Section to Microarray

Microarray for mRNA replication was prepared on the basis of <<1 M microarray>> from Agilent. Initial oligonucleotide microarray is prepared by chemical synthesis in situ. Oligonucleotides are attached to the surface by 3′ ends and have the following structure:

| | |
|---|---|
| #surf | 5′ tgtctcggAANNNNNNNNNNNNNNAGATCGGAAGAGCGTCG |
| | TGTAGGGAAAGAGTGTAGA 3′ |

3′ region (underlined) corresponds to the Illumina first sequencing adapter. (dN)₁₄ middle part of the oligonucleotide is a code. The code is unique for each feature of the microarray. Nucleotide sequences of codes were selected from 4¹⁴ theoretically possible variants using following criteria.
GC content: min 20%, max 66%.
purine (AG) or pyrimidine (CT) content: min 33%, max 66%.
purine (AT) or pyrimidine (CG) spots: max length 8 nt
homopolymeric spots: max length 4 nt
regions correspondent to common parts of the adapters, oligos for PCR or bridge amplification: max length 8 nt

Besides, those full-length #surf oligonucleotides were excluded from selection, which had at least 5 nt long regions correspondent to 3′ end parts of primers for PCR or bridge amplification.

Reactions on microarray were performed in Grace bio-labs hybridization chamber. Incubation at certain temperatures was done on glass slide adapter in MJ Research PCR machine. Washing in between the reactions was performed by 3 changes of the solution in hybridization chamber. Normally, reaction buffer for the next reaction was used for washing. Enzymes were purchased from New England Biolabs, unless otherwise specified. 0.1μg/μl BSA was added to all reactions to prevent non-specific sorption.

Oligonucleotide markers for mRNA capturing have the following structure:

| | |
|---|---|
| #surf | 5′ tgtctcggAANNNNNNNNNNNNNNAGATCGGAAGAGCGTCGTGTAGGGAAAGAGTGTAGA 3′ |
| #capt | 3′ TTTTTTTTTTTTTTTTTTTTacagagccTTNNNNNNNNNNNNNNTCTAGCCTTCTCGCAGCACATCCCTTTCTCACATCT 5′ |

Capturing oligonucleotides are attached to the glass because of hybridization to complementary #surf oligonucleotides. 5′ region (underlined) of capturing oligonucleotides corresponds to the Illumina first sequencing adapter. (dN)₁₄ middle part is a code. 3′ (dT)₂₀ end hybridizes to polyadenylated RNA. 8-nucleotide region preceding 3′ (dT)₂₀ end has no (dT) nucleotides and is used for preparation of sticky end by exonuclease activity of T4 polymerase.

Capturing oligonucleotides were prepared by several enzymatic reactions on microarray:
conservative 5′ part was attached to #surf oligonucleotides by hybridization;
code region was synthesised by primer extension;
sticky end for attachment of 3′ (dT)₂₀ end was generated by exonuclease activity of T4 polymerase;
3′ (dT)₂₀ end was attached by ligation.

Hybridization chamber was attached to the microarray and washing with 1× NEBuffer 2 was performed.

Hybridization with oligonucleotide #y008 was performed in (1× NEBuffer 2, 0, 1 μM of oligonucleotide #y008):
90° C.—5 min
0.1° C./sec to 50° C.
50° C.—15 min
0.1° C./sec to RT

| | |
|---|---|
| #surf | 5′ tgtctcggAANNNNNNNNNNNNNNAGATCGGAAGAGCGTCGTGTAGGGAAAGAGTGTAGA 3′ |
| #y008 | 3′ TCTAGCCTTCTCGCAGCACATCCCTTTCTCACATCT 5′ |

Primer extension was performed in (1× NEBuffer 2, 0.25mM dNTPs, Klenow 3′->5′ exo minus, 0.6 u/μl) for 30 min at 37° C., followed by 3 times washing with 1× NEBuffer 2.

T4 DNA polymerase digestion was performed in (1× NEBuffer 2, 1mM thio dTTPs, T4 DNA polymerase, 0.5 u/μl), for 20 min at 12° C., followed by 3 times washing with 1× NEBuffer 2 and one wash with 1×T4 DNA Ligase Reaction Buffer. Thio-modified nucleotides are resistant to T4 DNA polymerase. So, at this step the oligonucleotide duplexes on the surface of the microarray looked the following way:

| | |
|---|---|
| #surf | 5′ tgtctcggAANNNNNNNNNNNNNNAGATCGGAAGAGCGTCGTGTAGGGAAAGAGTGTAGA 3′ |
| | 3′ TTNNNNNNNNNNNNNNTCTAGCCTTCTCGCAGCACATCCCTTTCTCACATCT 5′ |
| #y006 | 5′ Phosphate-CCGAGACATTTTTTTTTTTTTTTTTTTT 3′ |

Oligonucleotides #y006 were ligated to the recessive 3′ ends in (1×T4 DNA Ligase Reaction Buffer, 0.1 μM of oligonucleotide #y006, 20 u/μl T4 DNA Ligase):
30° C.—5 min
0.1° C./sec to 16° C.
16° C.—60 min

Grace bio-labs hybridization chamber was removed from the glass slide. Capturing microarrays were washed 3 times with buffer (100 mM Tris-HCl, pH 7.5, 500 mM LiCl, 10 mM EDTA) and stored in this buffer at 4° C.

mRNA Replication

Prior to replication, 1mm thick 12% polyacrylamide gels attached to the glass slides were impregnated with Lysis/Binding Buffer (100 mM Tris-HCl, pH 7.5, 500 mM LiCl, 10 mM EDTA, 1% LiDS, 5 mM dithiothreitol (DTT), Dynabeads® mRNA DIRECT™ Kit, Ambion #61011). This buffer lyses the tissue and provides conditions for hybridization of mRNA to the Oligo(dT) capturing oligonucleotides.

10 μm thick cryosections of 14 days mouse embryo were placed on the capturing microarray. Slides were cooled to 0° C. Ice-cold polyacrylamide gel with Lysis/Binding Buffer was put over the slide with cryosection, so that the gel covered the section. Sandwiches were incubated at room temperature for 25 minutes, and then cooled to 0° C. and disassembled. Slides were washed with Washing Buffer A (10 mM Tris-HCl, pH 7.5, 0.15 M LiCl, 1 mM EDTA, 0.1% LiDS) and then with Washing Buffer B (10 mM Tris-HCl, pH 7.5, 0.15 M LiCl, 1 mM EDTA).

First strand synthesis and elution of cDNA were performed in Grace bio-labs hybridization chamber. After 3 times washing with 1× Reverse transcription buffer first strand synthesis was performed using First-Strand Synthesis System (Invitrogen, #18080-051), but with 5× excess of SuperScript® III.
15° C.—5 sec
0.1° C./sec to 25° C.
25° C.—10 min
0.1° C./sec to 40° C.
40° C.—45 min
0.1° C./sec to 50° C.
50° C.—15 min

After 3 times washing with (10 mM Tris-HCl, pH 7.5, 0.15 M NaCl, 1 mM EDTA) cDNA was two times eluted in 10 mM Tris-HCl pH 7.5:
95° C.—5 min
0° C. (on the ice-cold metal plate)

Size-selection was performed by purification of DNA/RNA duplexes with AM Pure RNA beads (Beckmann). RNAse H treatment was performed in 1× Reverse transcription buffer as recommended by Invitrogen (First-Strand Synthesis System, #18080-051). Random primer (#rp_ss on FIG. 12A, 1 μg per reaction) was extended on cDNA by Klenow exo (-) polymerase in 1× NEBuffer 2, at 25° C. for 10 min, 37° C. for 30 min:

| | |
|---|---|
| #rp_ss | 5′ GTGACTGGAGTTCAGACGTGTGCTCTTCCGATCTNNNNNN |
| | NNN 3′ |

5′ region of the #rp_ss oligonucleotide (underlined sequence) corresponds to the Illumina 2^{nd} sequencing adapter.

Preamplification and sequencing in paired-end mode on MiSeq was performed according to standard Illumina protocols. First read was 50 bp long, which was enough to determine the area-specific codes. Second read was 75 bp for reliable recognition of the transcripts.

After revealing of codes and corresponding transcripts, gene expression maps were generated for individual genes. On a panel with 1 million features each read was presented as a point, placed in a position of the panel according to the code associated with that read. For reads with the same positions points were placed near each other. Comparison of obtained maps with in situ hybridization images for the same transcript (Transcriptome Atlas Database for Mouse Embryo, available at http://www.eurexpress.org/) demonstrated the similarity of results obtained by spatially resolved transcriptome sequencing approach to the data obtained by in situ hybridization.

### Example 3

### Spatially Resolved Locus-Specific Transcriptome Sequencing

The spatially resolved transcriptome sequencing approach allows to perform whole transcriptome expression profiling. It is useful for hypothesis-free studies. However in hypothesis-driven studies and in clinical analyses expression of only particular genes is interesting for researcher. Other genes are useless and only decrease the sequencing efficiency. In this case it would be nice to restrict analysis to particular list of genes.

To perform spatially resolved locus-specific transcriptome sequencing it is possible to start from in situ hybridization of gene-specific probes to the fixed tissue sections. The structure of hybridization probes is shown on FIG. 13A. Middle parts of the probes correspond to particular transcripts. Flanking regions are common to all probes: 5′ region corresponds to the sequencing adapter; 3′ region is required for hybridization to the oligonucleotide markers.

Slide with tissue section is assembled into sandwich-like assembly with spatially coded microarray. Structure of the oligonucleotides on the microarray is shown on FIG. 13B. 3′ region of #capt is complementary to the 3′ region of the hybridization probes, spatial code is located in the middle part, 5′ end is covalently attached to the glass slide and corresponds to sequencing adaptor I. After denaturation of “mRNA—hybridization probe” duplexes by heating sandwich-like assembly, probes diffuse from the tissue section and hybridize to the microarray

After disassembling the slides, extension of hybridized probes along the oligonucleotide markers is performed. Extended products contain sequencing adapters on both sides. Preamplification is performed with standard Illumina PCR primers, and ready sequencing library molecules with full-length adapters are obtained. Analysis of obtained data is performed as in the Example 2. The difference is that the expression maps are generated for preselected genes only.

The advantages of described approach:
lower sequencing costs and simplified data analysis;
experimental procedure is more handy,
analysis may be performed not only for cryosections, but also for formalin fixed, paraffin-embedded tissue (FFPE tissue) sections. It is important because FFPE tissues are most common specimens in clinics.

### Example 4

### Spatially Resolved Locus-Specific Genotyping

The method for in situ expression profiling of specific genes described in Example 3 may be adopted for the analysis of DNA within a tissue sections. The only difference is that locus-specific probes should be hybridized with DNA and designed to take copies of a sequences of a particular genomic loci (such as probes for GoldenGate technology/Illumina/, FIG. 14). After primer extension and ligation probes would have the same structure as the hybridization probe in Example 3, and may be replicated, sequenced and analyzed in the same way.

Locus specific sequencing permits to analyze mutation status of a number of genes (for example, the state of oncogenes in a tumor) for all cells in a tissue section.

### Example 5

### Positional Coding with Replication of Two-Dimensionally Distributed Oligonucleotide Markers

mRNA molecules are transferred to a coded microarray for positional coding in the Example 2, FIG. 12. The same microarray was used for positional coding of transcripts in paraformaldehyde fixed (PFF) tissue sections with the opposite direction of replication: oligonucleotide markers are transferred to the tissue section (sample).

The procedure has been done as follows:
1. Preparation of a sandwich-like assembly of a microarray as described in Example 2 using a PFF tissue section;
2. Heating the assembly to denature the hybridized oligonucleotide markers on the microarray;
3. Incubation of the assembly at room temperature to give a possibility to the oligonucleotide markers containing oligo(dT) 3′ tails to hybridize to poly(A) tails of the mRNA in the tissue section;
4. Cooling down of the assembly to about 0° C. to slow down hybridization;
5. Washing the PFF tissue section to remove non-hybridized oligonucleotide markers;
6. Performing first strand synthesis in situ
7. Purifying of cDNA with spatial codes from PFF tissue section and preparing a sequencing library.

Cooling down the assembly (in step 3.) after denaturation (in step 2.) resulted in rehybridization of a significant part of oligonucleotide markers back to the microarray. But some part of the oligonucleotide markers diffused into the PFF tissue section and hybridized with the mRNA.

### Example 6

### Combinatorial Positional Coding using Two Types of Oligonucleotide Markers

Using two types of oligonucleotide markers for positional labeling gives a possibility to synthesize a smaller number of different known sequences (codes).

FIG. 15 shows how in situ genotyping reaction has been coded with two types of oligonucleotide markers.

Detector probes obtained as a result of primer extension and ligation are transferred to the coded surface for bridge amplification. Upstream genotyping oligonucleotides have conservative 5′ ends, correspondent to primers for bridge amplification type 1. Downstream genotyping oligonucleotides have conservative 3′ ends, complementary to primers for bridge amplification type 2 (FIG. 15A).

There are two types of primers for bridge amplification: type 1 and type 2. 5′ ends are conservative and correspond to the first and second sequencing adapters respectively. 3′ ends are conservative and correspond to upstream and downstream genotyping oligonucleotides adapters respectively. Middle parts are coding regions. The target surface is subdivided on rows and columns (X- and Y-coordinates respectively, FIG. 15B). Different columns have different type 1 codes (X-coordinates). Different rows have different type 2 codes (Y-coordinates).

After bridge amplification the detector probes have a structure as shown on FIG. 15A:
internal detector part gives a possibility to recognize the genomic locus and the allelic status of this locus;
code on type 1 end corresponds to the X-coordinate of position of the detector molecule on a tissue section;
code on type 2 end corresponds to the Y-coordinate of position of the detector molecule on a tissue section.

### Example 7

### In situ PCR with Spatial Coding

In situ PCR technology allows amplification in tissue sections. Amplification products localize in the same places, where templates were.

Combination of the in situ PCR technology with the idea of spatial coding described in this patent permits to analyze spatial distribution of many types of nucleic acids in parallel (due to the sequencing of the resulting products) with a high sensitivity and spatial resolution corresponding to that of in situ PCR.

Scheme of the in situ PCR with spatial coding (in situ SC-PCR) is shown in FIGS. 16A and 16B. In contrast to the classical in situ PCR:
tissue section is placed on a glass slide with oligonucleotide markers;
and (optionally) amplification is carried out in asymmetric mode with the reduced concentration of one of the primers (primer #b in FIG. 16).

On both schemes (FIGS. 16A and 16B) four oligonucleotides are used for in situ SC-PCR. Template #templ is amplified with primers #a and #b. Primer #code contains spatial code (drawn as a box) in the middle part, 3′ end of primer #code coincides with primer #b, 5′ end coincides with primer #c. Concentrations of primers #a and #c are normal for in situ PCR, concentration of primer #b is reduced. In situ amplification of #templ with #a and #b occurs in assymetric mode because of the reduced concentration of the primer # b. Spatial coding of amplification products is performed by extension of the chain, which is synthesized in excess (upper strand on the FIG. 16), along the coding oligonucleotide #code. Extended molecules with a code are further amplified in situ with primers #a and #c.

Primers #a, #b and #c are the same in all areas of the in situ PCR reaction, but primers #code should have very special spatial distribution. For successful spatial coding different #code oligos should be in each area of a glass slide. There are two variants how to provide specific distribution of the coding oligonucleotides # code in the reaction. In variant FIG. 16A oligonucleotides #code are attached to the glass slide, in variant FIG. 16B oligonucleotides #code are synthesized in situ.

In the variant shown in FIG. 16A coding oligonucleotides #code are attached to the glass slide. In the early PCR cycles amplification occurs as in the conventional in situ PCR. Template #templ is amplified with primers #a and #b. Primer #c is not used because there is no any complementary sequence for #c in the reaction. PCR runs in asymmetric mode because of the deficiency of primer #b. However, once the amplification products reach the glass with attached coding oligonucleotides, they get extended and may be further amplified with primers #a and #c available in sufficient amounts.

To catch a code in the scheme on FIG. 16A amplified molecules should contact the coding oligonucleotides fixed on a glass slide. In some cases, this requirement is too strict, so the scheme on FIG. 16B shows a variant in which the coding oligonucleotide #code is not attached to the glass slide. Oligonucleotides #code are produced locally by in situ primer extension from oligonucleotides fixed on the glass slide. Thus, in the early cycles of PCR in parallel to the conventional in situ PCR, linear amplification of the coding oligonucleotides attached to the glass slide takes place. As in the scheme on FIG. 16A on early cycles of PCR runs in assymetric mode because of the reduced concentration of primer #b. Also, primer #c has no targets in solution (only oligonucleotides attached to the glass are targets for #c). However, once the amplification products and synthesized primers #code meet each other amplification product is extended and amplification goes further in the normal mode with primers #a and #c.

In the case shown in FIG. 16A amplification products should reach coded glass, and in the case shown in 16B amplification products should meet with oligonucleotides synthesized on the glass. The permeability of tissue section for oligonucleotides may be higher, than that for PCR products, so the option “B” may be successful in some cases when option “A” does not work.

For the analysis of results of in situ SC-PCR it is necessary to isolate DNA from tissue section and to separate molecules which have regions #a and #c at the ends from all others (unused primers, non-coded amplification products with #a and #b regions at the ends, etc.). If the 5′ regions of the primers #a and #c correspond to the 3′ regions of the sequencing adapters, it would be enough to perform preamplification for preparation of the sequencing library. Thus, only amplified molecules with codes would be sequenced.

The in situ SC-PCR is especially useful for in situ genotyping (Example 4). Only few products of genotyping reaction (detector molecules) are obtained per locus in each cell after primer extension and ligation. Besides, some losses are inevitable during the transfer of detector molecules to the coding slide and preparation of the sequencing library. Amplification of detector molecules in tissue section is highly desirable.

The scheme of in situ genotyping with SC-PCR amplification is shown in FIG. 17. Reaction is performed in two steps. Detector molecules are produced after hybridization, primer extension and ligation. Second step is in situ SC-PCR. It is performed as described above—either with #code oligonucleotides attached to the glass slide (FIG. 16A) or with in situ synthesis of #code oligonucleotides (FIG. 16B). For in situ SC-PCR upstream genotyping probes should have conservative 5′ parts (coinciding with in situ PCR primer #a) and downstream genotyping probes—conservative 3′ parts (complementary to in situ PCR primer #b). Oligonucleotides #a, #b, #c and #code (shown on FIG. 17) are designed in the same way as on FIG. 16. Sequencing of amplified spatially coded detector molecules provides:
sequence of the detector part gives a possibility to recognize the genomic locus and the allelic status of this locus;
sequence of the coding part—position of the detector molecule on a tissue section.

Similarly, it is possible to use in situ SC-PCR for amplification of expression profiles. The scheme is shown in FIG. 18. Reaction is performed in two steps. On the first step RNA is reverse transcribed into cDNA. Second step is in situ SC-PCR. It is performed as described above—either with #code oligonucleotide attached to the glass slide (FIG. 16A) or with in situ synthesis of #code oligonucleotide (FIG. 16B). To be compatible with in situ SC-PCR, primers for the first strand synthesis should have conservative 5′ parts (complementary to in situ PCR primer #b). 3′ parts may be different, depending on the task of the researcher: oligo (dT), random primers, oligonucleotides for specific transcripts, etc. Oligonucleotides #a, #b, #c and #code (shown on FIG. 18) are designed in the same way as on FIG. 16. The only difference is that for amplification of cDNA it is necessary to use a combination of conservative primer #a with a set of primers with conservative 5 ‘part (coinciding with #a) and variable 3’ parts corresponding to different transcripts: random parts for unspecific amplification or a set of locus-specific parts for analysis of specific transcripts.

Sequencing of spatially coded transcripts provides following information:
sequence of gene-specific part gives a possibility to recognize a gene; the number of reads correspondent to a particular gene is proportional to the expression level of this gene.
sequence of the coding part—position of the transcript in a tissue section.

Taken together, sequencing permits to make a conclusion about expression level of particular genes in different areas of tissue section.

1-15. (canceled)

16. Method for identification of areas of a sample from which nucleic acid molecules originate using labeling of said nucleic acid molecules by two-dimensionally distributed oligonucleotide markers comprising the following steps:
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if nucleic acid molecules are not attached to the sample, providing conditions to minimize shift of nucleic acid molecules from the original positions on or within the sample; or
c) if nucleic acid molecules are attached to the sample, providing conditions for releasing the nucleic acid molecules;
d) assembling the sample and the target surface in such a way, that the distance from positions of said nucleic acids to the target surface is smaller than the distortion acceptable for a replica and with a medium in between sample and target surface;
e) providing conditions for diffusion of the nucleic acid molecules from the sample to the target surface and hybridization-based binding of the nucleic acid molecules to the oligonucleotide markers on the target surface;
f) releasing of hybrids of the nucleic acid molecules and the oligonucleotide markers into solution;
or
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if oligonucleotide markers are not attached to the target surface, providing conditions to minimize shift of nucleic acid oligonucleotide markers from the original positions on the target surface; or
c) if oligonucleotide markers are attached to the target surface, providing conditions for releasing of oligonucleotide markers;
d) assembling the sample of and the target surface in such a way, that the distance from positions of oligonucleotide markers to the sample is smaller than the distortion acceptable for a replica and with a medium in between sample and target surface;
e) providing conditions for diffusion of the oligonucleotide markers from the target surface to the sample and hybridization-based binding of the oligonucleotide markers to the nucleic acid molecules on the sample;
f) releasing of hybrids of nucleic acid molecules and the oligonucleotide markers into solution;
or
a) providing the sample containing nucleic acid molecules located either on the surface of the sample or within the sample;
b) providing a target surface with two-dimensionally distributed oligonucleotide markers with known sequences, wherein each marker corresponds to a defined area on the target surface;
c) if the oligonucleotide markers are not attached to the target surface and the nucleic acid molecules are not attached to the sample, providing conditions to minimize shift of the oligonucleotide markers and/or the nucleic acid molecules from the original positions; or
c) if the oligonucleotide markers are attached to the target surface and the nucleic acid molecules are attached to the sample, providing conditions for releasing of the oligonucleotide markers and/or the nucleic acid molecules;
d) assembling the sample and the target surface in such a way, that the distance from positions of said nucleic acids to the target surface is smaller than the distortion acceptable for a replica and with a medium in between sample and target surface;
e) providing conditions for diffusion of the oligonucleotide markers from the target surface and the nucleic acid molecules from the sample and hybridization-based binding of the marker oligonucleotides to the nucleic acid molecules within the medium between sample and target surface;
f) disassembling the sample and the target surface and collecting the medium containing hybrids of the nucleic acid molecules and the oligonucleotide markers; and
g) analyzing the hybrids in order to determine the nucleotide sequence of the oligonucleotide markers;
h) identification of the areas of the sample from which the nucleic acid molecules originated as areas correspondent to the positions of the oligonucleotide markers.

17. Method according to claim 16, wherein step c) is only performed after step d) (assembling of the sample and the target surface).

18. Method according to claim 16, comprising after step e) further step e):
e) providing conditions for slowing down the formation of new hybrids of nucleic acid molecules and marker oligonucleotides.

19. Method according to claim 16, wherein the nucleic acid molecules in the sample or the nucleic acid molecules on the target surface contain known sequences, which get inserted in the nucleic acid molecules from the target surface or nucleic acid molecules from the sample by primer extension or ligation reactions and said known sequences are further used for analysis of replicas, wherein said analysis may be performed on the target surface or in solution.

20. Method according to claim 16, wherein (i) the known sequences are different between the samples, the target surfaces, replication experiments and serve to distinguish the samples, the target surfaces, and/or replication experiments or (ii) wherein the known sequences are different in different regions of the sample or of the target and serve to determine the position of nucleic acid molecules on the target surface or in the sample.

21. Method according to claim 16, wherein the nucleic acid molecules located on the surface of the sample are distributed in a nucleic acid array or protein array, or wherein the nucleic acid molecules distributed within a sample are distributed in a gel layer, in tissue section, in cell or tissue array or in a block of tissue.

22. Method according to claim 16, wherein the two-dimensionally distributed oligonucleotide markers with known sequences are provided as microarray or as two-dimensionally distributed microbeads, covered with oligonucleotides.

23. Method according to claim 16, wherein the hybrids of the nucleic acid molecules and the oligonucleotide markers with known sequences are linked by ligation, by primer extension of oligonucleotide markers on nucleic acids, or by primer extension of nucleic acids on oligonucleotide markers.

24. Method according to claim 16, wherein the areas of a sample correspondent to different oligonucleotide markers are overlapping or isolated from each other.

25. Method according to claim 16, wherein the sample, the target surface or both are subdivided into isolated regions, wherein the nucleic acid molecules and the oligonucleotide markers can't cross the borders of the regions and wherein the regions are created by using a mask with isolated holes or by scratching the sample or the target surface.

26. Method according to claim 16, wherein the conditions for diffusion of the nucleic acid molecules or oligonucleotide markers in step e) are facilitated by liquid flow (blotting) or by electric field (electrophoresis).

27. Method according to claim 16, wherein one oligonucleotide marker is attached per nucleic acid molecule.

28. Method according to claim 16, wherein two types of oligonucleotide markers are attached to each nucleic acid molecule: one to the 3′ end and another one to the 5′ end.

29. Method according to claim 16, wherein the analysis of the hybrids is performed by sequencing.
